(19) United States
(12) Patent Application Publication
(10) Pub. No.: US 2002/0177120 A1
(43) Pub. Date: Nov. 28, 2002

(54) ASSAYS FOR APOTOSIS MODULATORS

(76) Inventors: KATHRYN J. ELLIOTT, SAN DIEGO, CA (US); MARIA Z. KOUNNAS, SAN DIEGO, CA (US); REBECCA J. DYER, SAN DIEGO, CA (US); BENITO MUNOZ, SAN DIEGO, CA (US); STEVEN L. WAGNER, SAN DIEGO, CA (US)

Correspondence Address: STEPHANIE L. SEIDMAN, HELLER EHRMAN WHITE & MCAULIFFE, 4250 EXCUTIVE SQUARE, 7TH FLOOR, LA JOLLA, CA, 920379103

(21) Appl. No.: 09/326,472

(22) Filed: Jun. 4, 1999

(51) Int. Cl. C12Q 1/00; C07K 2/00; C07K 4/00; C07K 5/00; C07K 7/00; C07K 14/00; C07K 16/00; C07K 17/00; A61K 38/00; C07K 1/00

(52) U.S. Cl. 435/4; 530/300; 530/350

(57) ABSTRACT

Recombinant cells expressing fluorescence resonance energy transfer reporter polypeptides and cell-based assays for apoptosis; screening assays for identifying and selecting candidate compounds modulating apoptosis.

### FIELD OF THE INVENTION

The invention relates generally to predictive assays for identifying and selecting compounds exerting effects on programmed cell death.

### BACKGROUND OF THE INVENTION

Growth, proliferation, differentiation and cell death are genetically programmed in eukaryotic cells to occur at different rates in different tissues. Energy-dependent programmed cell death, referred to as apoptosis, involves mechanisms that are distinguishable from necrotic cell death (1-4). Apoptosis is inducible by interaction of ligands with cell surface proteins, e.g., Fas ligand/Fas, TNFR1/TNF, Death Receptor 3/Apo 3 ligand and glycocorticoids, but also by destruction of cellular DNA and structures by radiation; or by removal of life-sustaining agents such as serum, glucose and/or growth factors. Induction of apoptosis is accompanied by intracellular changes in a cascade-like fashion, including changes in heterodimeric cysteine proteases. Apoptosis proteases reportedly hydrolyze a variety of life-critical intracellular proteins (2). Compounds that selectively affect apoptotic processes constitute valuable molecular probes useful in unraveling the multiple interactions leading up to the commitment of a cell to die. Inhibitors of cell death also find potential pharmaceutical uses in treating traumatic disease, stroke and acute diseases associated with premature apoptotic death of cells in tissues. Promoters of cell death find potential use in treatments for cancer. Thus, screening assays capable of identifying compounds modulating apoptosis constitute valuable scientific and pharmaceutical tools.

Fluorescence resonance energy transfer (FRET) involves a distance-dependent interaction between the electronic excited states of two dye molecules in which the excited emission energy from a donor fluorophore is coupled to excitation of an acceptor fluorophore, without emission of a photon. Coupling of the two fluorophores results in both decreased emission from the donor and increased emission from the acceptor. The distance at which energy transfer occurs between the two fluorophores in 50% of excited molecules is defined by the Förster radius, which is typically about 10-100 Å. FRET between linked green fluorescence protein (GFP) mutants as a reporter system was suggested by Cubitt et al. (6) and Tsien et al. U.S. Pat. Ser. No. 5,439,797. Heim and Tsien (7) reportedly linked two GFP mutants, Y66H/Y145F (BFP) and S65C, through a trypsin-cleavable spacer, and reported disruption of FRET following trypsin treatment.

Activation of members of the caspase family of cysteinyl proteases is a feature of apoptosis. The activities of these caspases, commonly assayed in cell extracts, have been used to measure apoptotic processes. However, such extracellular assays of protease activity do not appear to be reflective of actual intracellular apoptotic events. Cell-based assays for apoptosis involving colorimetric or fluorescent substrates are also problematic since entry of substrate compounds into cells can be variable and/or toxic. Recently Xu et al. (5) reported transient transfection of cells with plasmid DNA encoding a hybrid GFP-BFP protein consisting also of an 18 amino acid polyGly-Ser linker with an embedded DEVD sequence. Transient transfection of cellular monolayers with both the GFP-BFP reporter plasmid and a Rip encoding plasmid (an apoptosis inducer) reportedly resulted in rounding and release of apoptotic cells. Fluorescence activated cell sorter (FACS) analysis of cells harvested 24-36 hrs. after transfection with just the reporter plasmid reportedly exhibited two peaks of fluorescence intensity, while cells co-transfected with both the reporter and Rip plasmids exhibited only a single peak. The change in cellular fluorescence profile was interpreted as indicating a possible disruption of FRET between GFP and BFP.

There exists a need for a cell-based apoptosis assay in which the read-out of the assay is directly related to the activity of one or more intracellular caspase enzymes, and in which rapid and reproducible measurements may be made of the effects of unknown compounds on apoptosis. Disclosed herein are FRET-based screening assays useful for identifying compounds that modulate apoptosis.

### SUMMARY OF THE INVENTION

The invention provides recombinant apoptosis reporter cells expressing FRET reporter polypeptides useful in screening assays for identifying and selecting compounds that modulate apoptosis. The screening assays are capable of detecting intracellular apoptotic effects induced by compounds applied extracellularly in a microtiter assay format, with as few as 40-80,000 cells and within just 16-24 hrs after addition of an apoptosis inducer agent. The assays are specific, sensitive, stable and reproducible. Apoptotic changes in cells can be monitored without lysing or extracting cells, and in certain preferred embodiments more than 80% of the apoptotic cells remain adherent at the time of assay thereby reducing light scatter and background in the assay. Assay validation was achieved by screening more than 24,000 random compounds and identifying and selecting 19 acting to modulate apoptosis. Two of these compounds were subsequently shown to selectively inhibit apoptosis.

### BRIEF DESCRIPTION OF THE DRAWINGS

FIG. 1 depicts the 75 nucleotide, and corresponding 25 amino acid sequences, linking fluorophores in FRET-reporters. In FIG. 1A is depicted a GFP-BFP linker region disclosed in the prior art (PRIOR ART). In FIG. 1B is depicted the sequences of a trypsin-cleavable linker region disclosed in the EXAMPLES section below and termed “SAT”. In FIG. 1C is depicted the sequences of a linker region in the FRET apoptosis reporter termed “SgAT”, as disclosed further in the EXAMPLES section below. In FIG. 1D is depicted the sequences of a linker region in the control FRET reporter termed “SnAT”, as disclosed further in the EXAMPLES section below.

FIG. 2A depicts the change in fluorescence resonance energy transfer, as a function of time, from FRET reporter polypeptides expressed in an F9-1-13 FRET reporter cell undergoing apoptosis induced by anti-Fas antibody, as described further in the EXAMPLES section, below.

FIG. 2B depicts the change in caspase 3 enzyme activity measured in the FRET reporter cells of FIG. 2A as a function of time following induction of apoptosis with anti-Fas antibody.

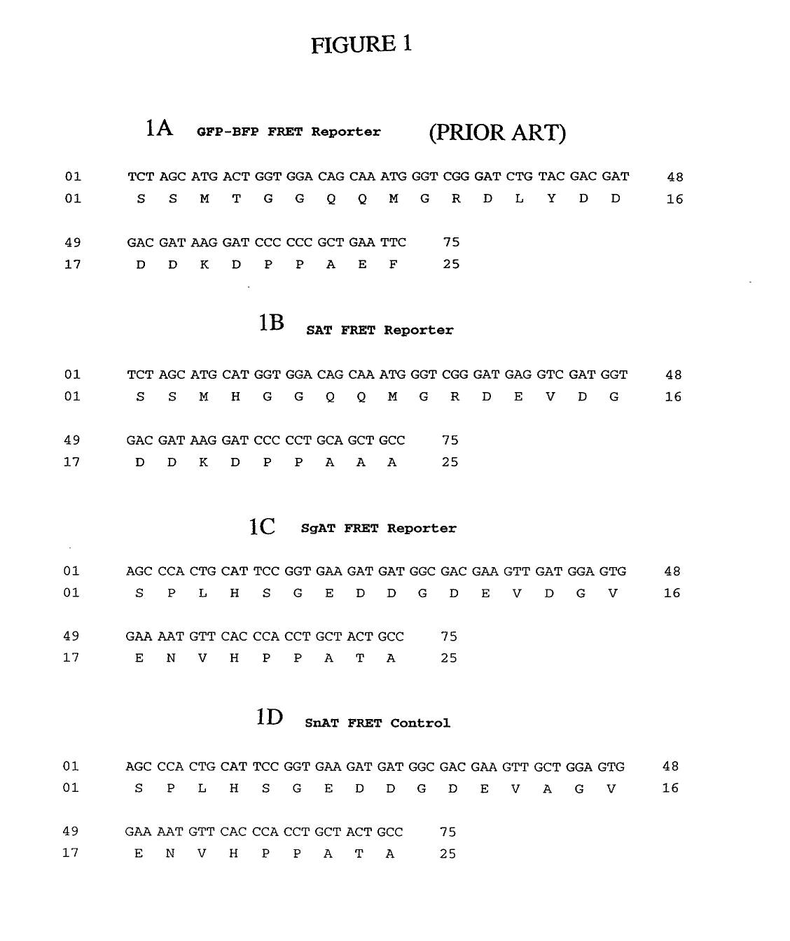

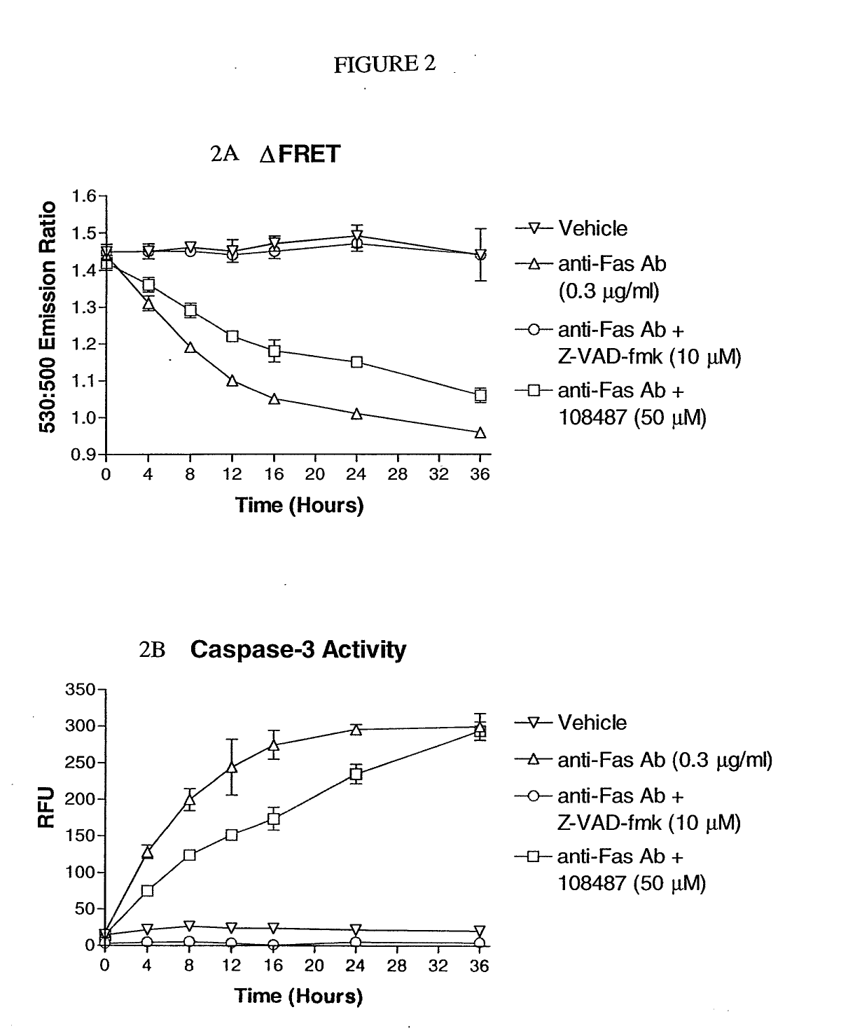

### DETAILED DESCRIPTION OF THE PREFERRED EMBODIMENT

As recited in the Background section above, Xu et al. (5) report transient transfection of cells with GFP-BFP plasmid DNA where the fluorophores were coupled through an 18 amino acid polyGly-Ser linker with an embedded DEVD sequence. DEVD is a potential substrate site cleavable by caspase-3.

The assays disclosed in Xu et al. were considered unsuitable for screening for at least the following six reasons: namely,

1. In Xu et al., cells harvested 24-36 hrs. after transient transfection with the FRET plasmid reportedly exhibited two peaks of fluorescence intensity in a fluorescence activated cell sorter; and cells co-transfected with both the reporter plasmid and a Rip (apoptosis-inducing) plasmid exhibited just a single peak. The change in cellular fluorescence was interpreted as disruption of FRET between GFP and BFP. However, under these conditions of transient transfection a homogenous cell population is not achieved. This is particularly relevant since the observed read-out of the assay is distant in time from the induction of apoptosis. If cells respond differently, or if not all of the cells in the cell population contain a reporter, then variability will necessarily be observed. Timing is also problematic since the length of time required for producing the reporter may differ from the time required for expression of the Rip inducer. Thus, the question of when to administer a test compound to the transfected cells becomes highly problematic. Under conditions of testing in screening assays where cells may be in the continuous presence of an unknown test compound, controlling intrinsic assay variability and timing was considered by the inventors to be a key to measuring effects induced by any extrinsically applied compound;

2. In Xu et al., apoptosis was induced by transient transfection with a Rip plasmid, i.e., again resulting in a non-homogeneous population of live and dying cells. Dying apoptotic cells eventually release their contents and these toxic byproducts can exert necrotic effects on adjacent non-transfected cells adding to assay variability unless this is controlled, e.g., as set forth below;

3. In addition to controlling variability, assay sensitivity was found (herein) to be key to achieving success in a screening assay (i.e., see the EXAMPLES section, below). Xu et al. does not disclose assay sensitivity or detection of FRET changes induced by an extracellular apoptosis inducer, e.g., TNF. Instead, possibly to increase signal strength, Xu et al. harvested floating (non-adherent) cells and analyzed cleavage of GFP-BFP by SDS-PAGE and western blotting.

4. Xu et al. fail to disclose the number of cells, or the assay volume, or the container size (e.g., wells, plates, flasks), or number of containers necessary to obtain a positive assay result. Since screening is preferably conducted in microplates using robotic pipettor and readers, relatively small numbers must produce a signal having a strength that is significantly greater than background, or the assay is not functional; and

5. Transient transfection is very labor-intensive, so much so that the inventors considered it to be unfeasible for use in high throughput screening; and,

6. Screening assays necessarily must be able to reproducibly assay tens of thousands of compounds with specificity, sensitivity and precision if they are to be used successfully to identify those few positive “hits” that may result in identification of a lead compound. Uncontrolled assay variations resulting from uncontrolled changes in cellular physiology can lead to identification of thousands of false-hits.

In view of the foregoing recognized defects in the art, it is believed unlikely that the methods disclosed by Xu et al. could be used successfully in screening. Independently, the inventors, recognizing the need and inherent problems, conducted a series of studies designed to determine empirically the requirements for FRET cell-based apoptosis screening assays. The results of those studies are disclosed in the EXAMPLES section below, and form basis for the embodiments of the invention as disclosed below.

As used herein the symbols for amino acids are according to the IUPAC-IUB recommendations published in Arch. Biochem. Biophys. 115: 1-12, 1966 with the following single letter symbols for the amino acids: namely,

| | | | |
|---|---|---|---|
| L, Leu, Leucine | V, Val, Valine | Y, Tyr, | D, Asp, Aspartic |
| | | Tyrosine | Acid |
| I, Ileu, | P, Pro, Proline | W, Trp, | E, Glu, Glutamic |
| Isoleucine | | Tryptophan | Acid |
| M, Met, | G, Gly, | N, Asn, | K, Lys, Lysine |
| Methionine | Glycine | Asparagine | |
| T, Thr, Threonine | A, Ala, | Q, Gln, | R, Arg, Arginine |
| | Alanine | Glutamine | |
| F, Phe, | S, Ser, Serine | C, Cys, Cysteine | H, His, Histidine |
| Phenylalanine | | | |

Other abbreviations used include: FRET, fluorescence resonance energy transfer; kDa, kilodaltons; ATP, adenosine triphosphate; LDH, lactate dehydrogenase; TNF, tumor necrosis factor; IL-1, interleukin 1; EGF, epidermal growth factor; PDGF, platelet derived growth factor; TGF, transforming growth factor; HTS, high throughput screening; NGF, nerve growth factor; BDNF, brain derived neurotrophic factor; CNTF, cilliary neurotrophic factor; PC, positive control; and, NC, negative control.

The terms used herein are intended to have the following meanings: namely,

“Apoptosis” is intended to mean the cascade of energy (ATP) dependent events triggered by an apoptosis inducer agent and leading to programmed cell death through mechanisms commonly involving intracellular caspase enzymes; commonly requiring about 12 to about 24 hrs.; and commonly involving cell death. In certain preferred embodiments the invention provides methods for assessing apoptosis prior to cell swelling, fragmentation and/or lysis. Mechanistically, during apoptosis dying cells fragment their DNA and become fragmented themselves into membrane-bounded apoptotic bodies. The released apoptotic bodies are ultimately subject to phagocytosis by immune cells. Where potentially toxic products resulting from apoptotic cell death are removed by phagocytes, death of a cell commonly does not result in death of adjacent cells. Preferably, the instant methods provide assays for assessing apoptosis in a dying population of cells where fewer than about 30% have undergone lysis and DNA release, and most preferably, where fewer than about 10% of the cells have undergone lysis and DNA release. Apoptosis is most definitively proven to have taken place by rescuing dying cells and bringing them back to a condition of growth by addition of an apoptosis inhibiting agent. The disclosure provided herein indicates that caspase-3 activity in dying cells in a high throughput screening assay, i.e., employing the methods of the invention, can be decreased through addition of certain selective non-toxic compounds present in a large library of random small organic molecules.

Apoptosis is recognized to play a fundamental role in cell development, tissue renewal; generating and regulating immune responses; and, preventing malignant transformation. Apoptosis has been implicated in the pathogenesis of an increasing number of diseases and may contribute to neuronal loss resulting from acute insults, such as ischemia, trauma or seizures, infarcts, and certain chronic neurodegenerative diseases including Alzheimer's disease. Aspects of neuronal apoptosis and necrosis are discussed by Choi, 1996 (11).

“Necrosis” is intended to mean a process of cell death which is not energy dependent; not involving the caspase enzyme system; and, commonly involves cellular swelling and lysis with release of cellular constituents into the extracellular medium, e.g., LDH, in a relatively rapid manner, e.g., on the order of minutes or hours. Often, rapid release of necrotic materials from dead and dying cells results in death of adjacent cells. Necrosis is recognized to play a fundamental role in inflammation; acute tissue destruction during inflammation; and excitotoxicity, i.e., neuronal cell death resulting from over stimulation of cells through cell-surface receptors such as glutamate receptors.

“Apoptosis induction” as used herein is intended to mean the process of triggering changes in a cell that, once initiated, will lead to programmed cell death. The subject process may be triggered for example either as result of the interaction of an “apoptosis inducer agent” with a cell surface protein molecule, or damage to cellular DNA or structures (e.g., mitochondria) e.g., by radiation, or by the removal of one or more critical growth factor from the cell environment. Representative examples of apoptosis inducer agents include: anti-Fas antibody or Fas ligand (binding to Fas); staurosporin; interleukins (e.g., IL-1 binding to IL-1Rβ; TNFα binding to TNFR1 and TNFR2); Apo 3 ligand (binding to Death Receptor 3); glycocorticoids (binding to cell surface glycocorticoid receptors); TRAIL, TRANCE, okadaic acid, NOC18 and the like. Other examples of apoptosis inducers include physical inducer agents, e.g., doses of ultrasound or radiation (e.g., UV, gamma-ray, X-ray and the like) effective to damage nucleic acid and/or life-sustaining cellular DNA and intracellular structures, e.g., mitochondria. Yet other examples of ways in which apoptosis may be induced include removal of life-sustaining agents such as serum, glucose and/or growth factors. Representative examples of critical growth factors include, but are not limited to, glucose, and in growth factor-dependent cell lines, EGF, PDGF, TGFβ, BDNF, NGF and the like. Representative indicators for induction of apoptosis in a cell include intermucleosomal DNA fragmentation and increased activity of caspase cysteinyl proteases, such as caspase 3, caspase 8, caspase 9 and the like.

“Proapoptotic agent” is intended to mean an intracellular gene product effective to promote one or more apoptotic events in a cell undergoing apoptosis. Representative examples of genes whose products promote apoptosis include Bax, Bid, Cifa, Btf and the like.

“Modulating”, where used to characterize an effect of a compound on apoptosis induced in a population of cells, as measured in one or more assays, is intended to mean that the subject compound is effective to promote, or alternatively, to inhibit, one or more ongoing biochemical apoptotic events in a cell, wherein the subject apoptotic event is triggered by an apoptosis inducer agent. The resultant promoting, or inhibiting, may be indicated by an increase or decrease, respectively, in one or more of the following: namely, the number or percentage of cells within the population that are committed to cell death; the rate at which cells in the population are committed to die; or, the measured activity of one or more indicators of apoptosis. Representative indicators of apoptosis include, but are not limited to, an increase in the measurable enzymatic activity of one or more of caspase enzymes; an increase in internucleosomal DNA fragmentation; a decrease in mitochondrial oxidative energy metabolism; and/or, a decrease in the activity of biosynthetic enzymes, and the like.

“Modulate apoptosis”, when used with respect to characterizing a measured activity of one or more of the instant test compounds in the instant assay, is intended to mean any of the following: namely, (i) that the subject test compound attenuates the ability of an apoptosis inducer agent to trigger apoptosis in a cell population, e.g., as measured pretreating the cells with the subject test compound, adding an apoptosis inducer agent and then by determining the percentage of cells in a cell culture which are apoptotic about 16-24 hrs. after addition of an apoptosis inducer agent; (ii) that the subject test compound alters the rate of progression of apoptosis in a population of cells, e.g., as determined by making measurements with time of the extent of apoptosis in a cell population; and/or (iii) that the subject test compound alters the extent of apoptosis in a population of cells, e.g., as determined by evaluating the number of cells surviving about 16-24 hrs. after adding an apoptosis inducer agent.

“Interleukin” is intended to mean an agent released by a first cell, e.g., a cell of the immune system, that exerts an effect on a biological activity in a second cell, e.g., a neural cell. Representative interleukins include cytokines such as tumor necrosis factors (e.g., TNFα and TNFβ), IL-1 and the like.

“Growth factor” is intended to mean an agent capable of stimulating progression of cells through the cell cycle and resulting in an increase in cell number in a population of cells. Alternatively, growth factor is intended to encompass polypeptide factors required to maintain non-dividing differentiated cells (e.g., neurons) in a viable state. Representative growth factors include nerve growth factor (NGF), epidermal growth factor (EGF), platelet derived growth factor (PDGF) and the like.

“Linker polypeptide” is used to refer to a polypeptide resident within a FRET reporter polypeptide that is bonded to both an exciter fluorophore polypeptide and a emitter fluorophore polypeptide. Preferred linker polypeptides are composed of a serial array of amino acid residues wherein each amino acid is peptide bonded to its neighboring amino acid, and wherein the total length of the subject polypeptide (a) falls within the Förster distance of an exciter and a emitter fluorophore; and, (b) maximizes the opportunity for binding and cleavage by one or more apoptosis proteases. Presently preferred linker polypeptides are composed of about 18 to about 40 amino acids, preferably about 18 to about 30, and most preferably, about 18 to about 25 amino acids. In certain presently preferred embodiments, the subject linker polypeptide consists of the following amino acid domains: namely, (1) an end-cap domain containing end-cap amino acids; next, in serial array, (2) an acidic domain containing acidic amino acids; next, (3) a substrate domain containing a substrate for a protease; next, (4) a non-interfering domain, preferably containing acidic, neutral and/or non-polar amino acids but, overall, when assembled not comprising net hydrophilic or hydrophobic properties to the subject domain; and, last (5) a second end-cap domain containing end-cap amino acids.

“End-cap” amino acids, also referred to herein as “helix-breaking” and “helix end cap” amino acids, when used in regard to the instant “linker” polypeptide, is intended to mean those natural and unnatural amino acids that introduce bends into a polypeptide chain and, in nature, are found in regions of sequence adjacent to, but not in, alpha helices. Representative examples of end cap amino acids include Pro, Gly and Tyr.

“Helix-forming” amino acids are disclosed by Chou and Fasman (Advances in Enzymology 47: 45-148, 1978; Table 1, page 51), and subsequently by O'Neil and DeGrado (Science 250: 646-651; Table 2, page 650), as amino acids commonly participating in the formation of polypeptide alpha helix. As referred to herein “helix forming” is intended to mean strong “H_{α}“ helix formers as set forth in Chou and Fasman, supra, and amino acids having P_{α} values >1.4 at set forth in O'Neil and DeGrado (Table 2, page 650); Ala, Leu, Phe and Met are representative helix-forming amino acids.

“Non-interfering” amino acids, when used in regard to the instant “linker” polypeptide, is intended to mean that the subject amino acids do not interfere with cleavage of a substrate domain located within the subject linker polypeptide. Further, the subject non-interfering amino acid is either a natural (i.e., product of nature) or an unnatural amino acid commonly having an alkyl side chain, i.e., a side chain composed of hydrogen and carbon, and preferably, about about 1 to about 5 carbons; representative examples include combinations of Ala, Val, Ile, Leu, Gly, Ser, Thr, Cys, Tyr, Asn and Gln that are effective (in toto) not to interfere with cleavage of a substrate domain, and preferably, in toto are not hydrophilic or hydrophobic.

“Acidic” amino acids, when used in regard to the instant “linker” polypeptide, is intended to mean a natural or an unnatural amino acid having a side chain containing a carboxylic acid residue; representative examples include Asp and Glu.

“Hydrophilic” when used in regard to the “non-interfering” domain of the instant “linker” polypeptide, is intended to mean that the subject domain consists of amino acid residues that, when assembled into a peptide having about 3 to about 7 amino acids, do not confer hydrophilic properties on the subject peptide, e.g., water solubility at high concentrations of the subject peptide, e.g., solubility at >100 mg/ml in water.

“Hydrophobic” when used in regard to the “non-interfering” domain of the instant “linker” polypeptide, is intended to mean that the subject domain consists of amino acid residues that, when assembled into a peptide having about 3 to about 7 amino acids, do not do not confer hydrophobic properties on the subject peptide, e.g., water insolubility at low concentrations, e.g., insolubility at >1 mg/ml in water.

“Cassette”, when used in reference to a FRET reporter plasmid capable of encoding a FRET reporter polypeptide, is intended to mean that region of the subject plasmid which is capable of encoding the instant linker polypeptide, and preferably, that region within the subject plasmid that is capable of encoding the instant “substrate domain” portion of the instant linker polypeptide. Preferably, the cassette region of the plasmid is bounded on each side by restriction endonuclease sites, i.e., regions of nucleotide sequence cleavable by one or more restriction endonuclease, and preferably the subject restriction endonuclease sites are useful for orienting the direction (5′ to 3′) of an oligonucleotide that is to be inserted at the cassette site.

“In vitro cell-based assay method” is intended to mean an assay preferably conducted using a population of adherent cells to which an apoptosis inducer agent is added, or alternatively, conducted using a population of non-adherent cells to which an apoptosis inducer is added. Specifically excluded are assays in which non-adherent dead and dying cells are collected from a culture of initially adherent cells.

“Expression” and “expressing” when used herein in regard to the instant stable FRET reporter cell lines, and the instant FRET reporter polypeptide synthesized therein, is intended to mean that the subject cells constituting the cell line are capable of transcribing, translating, processing and retaining in intracellular stores the instant FRET reporter polypeptide in a form that comprises a peptide bond in a substrate site that is hydrolyzable by an intracellular enzyme induced during apoptosis, e.g., a caspase. That a cell line is so capable of expression may be determined empirically e.g., according to illustrative methods set forth in the EXAMPLES section, below.

“Identifying” when used in regard to one or more of the instant assay methods, is intended to refer to the process by which a random test compound, i.e., not known to modulate apoptosis, when subject to the conditions of evaluation imposed in the instant assay is observed to alter a FRET reporter activity, thereby determining that the compound is a “modulator of apoptosis”, as defined supra. “Identifying” within the context of the invention is envisaged to include assays involving both analyses of single test compounds as well as analyses in which more than one compound are included in an assay well, e.g., combinatorial approaches, wherein comparison of results obtained with different combinations yields the identity of a test compound effective as a modulator of apoptosis.

“Selecting” when used in regard to one or more of the instant assay methods, is intended to refer to the process by which a test compound, having been subject to, and resulting from a process of “identifying”, supra, is subsequently picked from within a group of test compounds for further study as a candidate compound.

“Candidate compound” is intended to mean a compound “identified” and “selected”, supra, for its activity in the instant assay. Candidate compounds are commonly subject to additional analysis, e.g. in secondary assays formats, to confirm that that they are active in modulating apoptosis, as defined supra.

“Exciter” fluorophore, when used in regard to the instant FRET reporter, is intended to mean that the subject fluorophore is capable of both (i) being excited by a fluorescent light and (ii) generating a resonance energy capable of exciting an emitter fluorophore located in the same FRET reporter molecule;

“Emitter” fluorophore, when used in regard to the instant FRET reporter, is intended to mean that the subject fluorophore is both (i) capable of receiving resonance energy generated by an exciter fluorophore and (ii) generating a fluorescent emission signal in response to the received resonance energy.

“FRET signal” is intended to mean the ratio of fluorescence emission signals recorded, after subtracting background, e.g., at 530 nm and 500 nm (i.e., abbreviated 530:500 nm) or at 530 nm and 495 nm (i.e., abbreviated 530:495 nm). The subject recorded emission signals being produced by the instant FRET-reporter emitter and exciter fluorophores as expressed in vitro in a recombinant cell after that cell is exposed to a fluorescent light source capable of activating the subject exciter fluorophore.

Embodiments of the invention solve the problems in the art by providing assays capable of detecting and quantifying intracellular apoptotic events that are induced following interaction of an apoptosis inducer agent with a cell, and assays in which:

(i) the instant stable recombinant FRET reporter cell lines were selected expressing sufficiently high levels of FRET reporter polypeptide on a per cell basis, and on a per cell population basis, that a FRET signal is detected and changes in the FRET signal are reproducibly induced following interaction of apoptosis inducer agents with the cell surface;

(ii) relatively small numbers of the instant FRET reporter cells, i.e., as few as 20 to 300 thousand per square centimeter, preferably about 60 to about 120 thousand per square centimeter, most preferably to achieve a robust signal in the assay about 120 to about 250 thousand per square centimeter. The subject cell numbers produce a sufficient precise, specific and sensitive FRET signal that apoptotic events are detectable within as little as about 16 hrs. to about 24 hrs, preferably in about 10 hrs. to about 16 hrs. Assay devices, e.g., microtiter plastic plates, for maintaining the instant FRET reporter cells as adherent cells during the course of the instant assay are preferably produced by treating the surface with cell adhesive ligands, e.g., collagen (i.e., gelatin), fibronectin and the like. Preferably, the instant FRET reporter cells remain adherent during the course of the assay and the instant assay duration is sufficiently short in time that many cells are still (marginally) alive and early enough in the apoptotic process that changes in cell microfilaments, microtubules, and adhesive molecules contributing to maintenance of cell shape are minimized, and rounding and release of cells from the substata is also minimized. Most preferably, greater than about 80% of the total cells in the assay are adherent at the conclusion of an assay when a FRET signal is determined. Skilled artisans may of course take a series of kinetic measurements to determine the rate of progression of FRET signal in the instant reporter cells during the course of an assay;

(iii) results obtained in the instant assays show that intracellular changes in FRET signal from the instant FRET reporter polypeptide are a direct consequence of cellular events triggered following addition of an apoptosis inducer agent to the assay;

(iv) the instant assay may be conducted with minimal intra-assay, inter-assay and day-to-day variation in signal using the instant stable FRET reporter cells;

(v) the instant stable FRET reporter cells minimize possible assay variability resulting from uncontrolled changes such as may result from changes in cell physiology, necrosis, transient transfection efficiency, selection in toxic media and the like;

(vi) precision and reproducibility were achieved in the instant assay without requiring use of large numbers of assay wells, i.e., to improve statistical significance, and commonly only single or duplicate microtiter assay wells were sufficient to record meaningful and reproducible results; and,

(vii) precision and reproducibility of the instant assay methods allowed identification of compounds modulating apoptosis at a low level, i.e., just a 7% to 10% difference in FRET signal between experimental (apoptosis-induced and treated with a test compound) and a positive control (apoptosis-induced only) was commonly significant, reproducible and basis for selecting compounds capable of modulating apoptosis. Specifically, following induction of apoptosis the FRET signal in positive control cultures (apoptosis-induced) preferably decreases by about 20% to about 50% from the non-apoptosis-induced values. Most preferably, the observed decrease in FRET signal following induction of apoptosis is about 25% to about 50%, and the choice of apoptosis inducer agent and the time at which the FRET signal is measured are adjusted to achieve these final results in control experiments. A significant inhibition of apoptosis was evidenced by a difference of as little as 7% between the FRET signal of positive control (apoptosis-induced) and experimental (apoptosis-induced+test compound). For example, if the apoptosis-induced control FRET signal is 50% then a decrease to a value of 43% (i.e., a 14% change) in experimental is commonly reproducible and sufficient to select a compound as a modulator of apoptosis; and, e.g., if the apoptosis-induced positive control FRET signal is 35% then a decrease to a value of 28% in experimental (i.e., a 20% change) is reproducible and sufficient to select a compound. Thus, greater than about a 10% change, i.e., an increase or decrease relative to the positive control, preferably greater than about a 20% change, is sufficient to select compounds capable of modulating apoptosis.

Embodiments of the invention provide an in vitro cell-based functional assay method for identifying and selecting candidate compounds capable of modulating apoptosis. Importantly, the use of an extrinsically applied apoptosis inducer agent in the instant assays more closely mimics, (i.e., than other available assays), the circumstances in which apoptosis is induced in ischemia, trauma, stroke and the like.

In other embodiments, the invention provides methods for preparation of a variety of different stable FRET reporter cell lines. The instant method involves the steps of: (i) inserting the instant FRET reporter plasmid containing a selectable marker into cells; (ii) selecting for the selectable marker, thereby selecting cells having incorporated the instant FRET reporter plasmid; (iii) testing the selected cells to insure that FRET reporter polypeptide is (a) synthesized by the cell, and (b) capable of producing a FRET signal that is at least about 3 to about 5-fold greater than the background signal produced in a control cell lacking the FRET reporter; (iv) further testing the selected cells to determine that (a) the FRET reporter polypeptide is cleaved following addition of an apoptosis inducer agent to a culture of the cells; and, (b) that FRET signal resident in the selected cells decreases by at least about 10% to about 30% within 24 hours after induction of apoptosis.

The instant assay methods involve the use of the instant stable recombinant FRET reporter cell lines that are determined empirically to be capable of expressing the subject FRET reporter polypeptide in an intracellular form that constitutes an effective substrate for intracellular proteases induced during apoptosis, e.g., caspases. Selection of the instant stable recombinant FRET reporter cell lines is based on the determination that addition of an apoptosis inducer agent to a culture consisting of about 120,000 to about 250,000 test cells per square centimeter results in a measurable decrease of at least 10%, preferably about 15% to about 50% in a FRET fluorescent signal produced by the test culture. The instant selection method empirically discards cell lines that do not properly transcribe, translate, process and place FRET reporter polypeptide into the cytosol or widely dispersed intracellular storage compartments that are accessible to proteases induced during apoptosis. Microscopic examination preferably reveals that the instant FRET reporter is relatively evenly distributed throughout the cytoplasm of the instant FRET reporter cell, and the instant reporter is not discretely localized in intracellular vacuoles or vesicles.

In other embodiments the invention provides expression plasmids capable of encoding intracellular FRET polypeptide substrates that consist of nucleic acid capable of encoding all of: (i) an exciter fluorophore, (ii) a linker polypeptide and (iii) an emitter fluorophore. The latter fluorescence exciter fluorophore consists of a fluorophore having an emission wavelength maximum that overlaps with the excitation wavelength profile of the emitter fluorophore. Preferably, the exciter and the emitter fluorophores have wavelength maxima for emission and excitation, respectively, which are within about 0 nm to about 30 nm of one another. In this manner the transfer of fluorescence resonance energy between the exciter and the emitter is maximized, and the sensitivity of the resultant assay is increased. Representative examples of exciter and emitter fluorophores so active include the mutant fluorophores disclosed according to methods disclosed in Tsien et al. U.S. Pat. Ser. No. 5,625,048 ('048) and also in published PCT/US96/01457 (WO97/28261; the '457 application). Two representative examples of exciter fluorophores are disclosed in the '048 patent at column 8, in TABLE I, “P9” and “P11” both having excitation maxima at about 396 nm and emission maxima at about 507 nm, both which are similar to the “Sapphire” exciter fluorophore disclosed in the Examples section, below. Representative emitter fluorophores include the “Topaz” exciter fluorophore disclosed in the Examples section below. Pollok and Heim (9) have recently reviewed uses of GFP in FRET-based applications and disclose certain properties of “Sapphire”. Commercially available exciter and emitter fluorophores, (and plasmid vectors including same), include enhanced blue fluorescent protein (EBFP) and enhanced green fluorescent protein (EGFP), respectively, (Clontech, Palo Alto, Calif.); and “Emerald” and “Topaz” (Packard Instrument Co., Meriden, Conn.).

TABLE I
Spectroscopic Properties of GFP and Related Mutants.

| | | | Molar | |
|---|---|---|---|---|
| | | | Extinction | |
| | Absorbance | Emission | Coefficient | Quantum |
| | Maximum (nm) | Maximum (nm) | (M⁻¹ cm⁻¹) | Yield |
| GFP¹ | 395² | 508 | 21,000-30,000 | 0.72-0.85 |
| BFP³ | 381 | 445 | 14,000 | 0.38 |
| S65C⁴ | 479 | 507 | * | * |
| Sapphire | 395 | 511 | 33,000 | 0.60 |
| Emerald | 487 | 508 | 36,000 | 0.70 |
| Topaz | 514 | 527 | 48,000 | 0.63 |
| EBFP⁵ | 380 | 440 | * | * |
| EGFP⁵ | 488 | 507 | * | * |

¹Cubitt et al. (6);
²GFP also has a minor absorbance peak at 475 nm;
³Heim and Tsien (7);
⁴Heim et al. (8);
⁵Clontech;
* Not reported.

The subject exciter fluorophores are preferably selected from among fluorescent polypeptides having fluorescence absorbance maxima of about 370 nm to about 490 and emission maxima of about 440 nm to about 514 nm, while the subject emitter fluorophores are selcted from among fluorescent polypeptides having fluorescence absorption maxima of about 470 nm to about 515 nm and emission maxima of about 500 nm to about 530 nm. Coupling of FRET signal between the exciter and emitter is achieved by selecting fluorescent polypeptides having properties as follows: namely, the emission wavelength maximum of the instant exciter fluorophore is within about 0 nm to about 50 nm, preferably, about 0 nm to about 30 nm, and most preferably, within about 0 nm to about 10 nm of the excitation wavelength of the instant emitter fluorophore; and also, the emission wavelength maximum of the emitter fluorophore is at least 10 nm to 20 nm greater than the emission wavelength of the exciter fluorophore. Illustrative combinations of exciter and emitter fluorophore combinations are set forth in TABLE II, on the following page.

TABLE II
Exciter-Emitter Combinations

| EXCITER | | | EMITTER | | |
|---|---|---|---|---|---|
| Absorb- | | | Absorbance | | |
| ance | | Emission | Max (nm) | | Emission |
| Max (nm) | Exciter | Max. (nm) | [Difference]* | Emitter | Max. (nm) |
| 395 | Sapphire | 511 | 514 [3] | Topaz | 527 |
| 395 | GFP | 508 | 514 [6] | Topaz | 527 |
| 479 | S65C | 507 | 514 [7] | Topaz | 527 |
| 487 | Emerald | 508 | 514 [6] | Topaz | 527 |
| 488 | EGFP | 507 | 514 [7] | Topaz | 527 |
| 381 | BFP | 445 | 479 [34] | S65C | 507 |
| 380 | EBFP | 440 | 479 [39] | S65C | 507 |
| 381 | BFP | 445 | 487 [42] | Emerald | 508 |
| 380 | EBFP | 440 | 487 [47] | Emerald | 508 |
| 381 | BFP | 445 | 488 [43] | EGFP | 507 |
| 380 | EBFP | 440 | 488 [48] | EGFP | 507 |

*[Difference] = Absorbance Max. of Emitter − Emission Max. of Exciter.

In other embodiments the invention provides FRET reporter plasmids encoding, and stable FRET reporter cell lines expressing, FRET reporter polypeptides that have an exciter fluorophore joined to a emitter fluorophore through a linker polypeptide. In preferred embodiments the linker polypeptide consists of about 18 to about 40 amino acids, preferably about 18 to about 30, and most preferably, about 18 to about 25 amino acids. The length of the instant linker polypeptide and the choice of its constituent linker amino acids are determined according to two requirements: namely, (1) that the linker join the exciter and emitter fluorophores at a distance which allows fluorescence resonance energy transfer from the exciter to the emitter, i.e., the length of the linker falls within the Förster radius of the exciter and emitter fluorophores; and, (2) that the linker contain a substrate domain amino acid sequence sufficiently long, and sufficiently optimized in amino acid sequence, for binding and cleavage by one or more apoptosis proteases. Apoptosis proteases exhibit a lesser apparent binding affinity (e.g., higher Km values) for small synthetic peptides than for larger, and a lesser binding affinity for amino acid sequences that are more divergent from native substrates, than those peptides which are more similar to native substrates. Thus, amino acid composition in and around the instant substrate domain is important for recognition by an apoptosis protease. The instant linker polypeptide comprises a serial array of domains selected to favor binding and cleavage by proteases induced during apoptosis. In presently preferred embodiments the linker polypeptide consists of a first end-cap amino acid domain, followed in order by, an acidic domain, a substrate domain, a non-interfering domain, and a second end-cap domain. Overall, the preferred length of the instant linker consists of about 18 to about 40 amino acids, preferably about 18 to about 30, and most preferably, about 18 to about 25 amino acids, as detailed further below.

Embodiments of the invention provide in vitro cell-based assays useful for screening unknown compounds to identify those which modulate apoptosis induced by an extracellular apoptosis inducer agent, or apoptosis induced by removal of a critical factor required for cell survival. In alternative embodiments, the instant methods provide cell-based assays for determining the structure resident within a test compound which is required for modulating apoptosis, i.e., structure activity response determinations of drug candidates useful for improving potency and advancing a drug candidate to the status of a clinical candiate. In yet other alternative embodiments, the instant methods are useful in screening for novel apoptosis inducer agents, e.g., compounds that may be useful anti-cancer agents. The instant assays are conducted by comparing the results obtained in three separate cultures, as described further below.

First, positive control cell cultures (PC) are established, e.g., by seeding about 40,000 to about 100,000 cells from a FRET reporter cell line into each of two wells in a 96 well microtiter plate, (or smaller numbers of cells for smaller plates); preferably the cells are adherent and will attach to the plastic substrata and grow. To facilitate adherence to the plastic substrata in the plate, those skilled in the art will recognize methods for improving adherence of cells to plastic substrata, e.g., using collagen coatings (e.g., gelatin or type I bovine collagen), fibronectin, loose fibrinogen clots, poly-L-lysine coatings and the like. After incubation to allow adherence of the cells, i.e., about 3 to about 5 hrs., each well of the subject 96-well microtiter culture preferably contains about 40,000 to about 100,000 cells, the cells are capable of expressing intracellular FRET reporter polypeptide, and at this point the PC cultures are ready for induction of apoptosis. Apoptosis is induced by adding an extrinsic apoptosis inducer agent such as staurosporin, or anti-Fas antibody, or by removing a critical component, e.g., glucose, from the cell's growth medium.

Second, a normal negative control cell culture (NC) is established in the same manner as with the PC culture, above, and using cells from the same FRET reporter cell line (above), the same amount of any vehicle, e.g., 0.25% DMSO, but without inducing apoptosis. Also, the addition of any vehicle to the NC is made at about the same time the test compound is added to the Experimental cultures, below.

Third, a number of different experimental cell cultures (Experimental) are established in the same manner as in the PC culture, above, including induction of apoptosis, except that each of the different Experimental cultures is brought into contact with a with a different compound (test compound) that is to be tested for its ability to modulate apoptosis. Those skilled in the art will recognize that it may prove desirable to add different concentrations of each different test compound to different cultures of cells, and that the test compounds may be added at different times prior to or following the induction of apoptosis. Preferably, test compounds are added at a final concentration of about 1 nM to about 300 μM, and most preferably, at a final concentration of about 10 μM to about 50 μM.

Next, the PC, NC and Experimental cultures, as established above, are cultured under conditions conducive to, and for a time sufficient to, induce apoptosis in the PC culture, e.g., as detected measuring DNA fragmentation or by measuring increased activity of one or more intracellular apoptotic proteases. Commonly, this process involves incubating cells in the PC, NC and Experimental cultures at 37° C., in a humidified atmosphere of 5% CO₂/95% air, in tissue culture medium containing growth factors and nutrients, such as, 5% fetal bovine serum, non-essential amino acids, glutamine and the like, for about 10 hours to about 24 hours, preferably about 16 to about 24 hrs. and most preferably about 16 to about 18 hrs. The selection of the subject assay duration is made after consideration of the rate at which apoptosis progresses after addition of a particular desired apoptosis inducer agent, i.e., as commonly determined by measuring a decrease in FRET signal according to the methods of the invention. In addition, microscopic observation will preferably show that greater than about 85% to about 95% of the cells in the subject cultures are adherent, and if a large number of non-adherent (floating) cells are observed the assay is discarded and conditions are altered, e.g., by setting up new assay cultures and using either a shorter incubation time or a lower concentration of the subject test compound.

To measure the intracellular energy transfer within the endogenous FRET reporter polypeptides in the cell cultures comprising the PC, NC and Experimental cultures, each culture is exposed to a fluorescent light source capable of exciting the endogenous exciter fluorophore and emission from both the exciter and the emitter fluorophore are measured, i.e., preferably sequentially within about 1 to about 3 minutes, and also preferably at two different selected wavelengths where emission from the exciter and emitter can be distinguished. FRET signal (defined supra) is calculated from the relative measured fluorescence units as the ratio of the emission of the emitter divided by the emission of the exciter. If the instant linker polypeptide in the intracellular FRET reporter polypeptide has been cleaved by a protease whose expression was induced, or whose enzyme activity was activated, during apoptosis, then the reporter polypeptide does not properly transmit resonance energy from the exciter to the emitter and the signal is decreased. Thus, to verify that apoptosis has been successfully induced in the assay a comparison is made between the FRET reporter emission (or FRET signal ratio) recorded from the PC and NC cultures. Preferably, the difference in the FRET signal (ratio) recorded from the PC culture is at least 20% to 50%, and preferably about 30% to about 50%, less than that in the NC culture, and preferably the NC culture measured fluorescence emission is at least about 3-fold to about 5-fold greater than the emission recorded in wells lacking cells, or wells containing cells that do not contain a FRET reporter (i.e., NC emission is at least 3-5 fold greater than background). If these conditions are met, then the result recorded in the experimental cultures is next examined.

Fluorescence measurements are made, as above, for each of the Experimental cultures. Apoptosis modulators, as defined supra, may promote or inhibit events triggered following induction of apoptosis. A test compound which inhibits apoptosis may be identified by a FRET signal (ratio) in an Experimental culture which is greater than that recorded in a PC culture; and a test compound which promotes apoptosis may be identified by a FRET signal (ratio) in an Experimental culture which is less than that recorded in the NC culture, provided these cultures do not exhibit evidence of necrotic/toxic cell death or fluorescence emission from the test compound. The determination that a test compound is a “candidate compound”, i.e., one selected as being worthy of further study as a probable apoptosis modulator, is based on the foregoing comparisons of emission signals recorded in the PC, NC and Experimental cultures, and the findings of an increase or decrease in fluorescent FRET emission signal recorded from the Experimental culture relative to the PC or NC culture, i.e., as described supra.

The results obtained with replicate PC and NC cultures in an assay may differ by as little as about 20% to about 50%, but preferably by about 30% to about 50%. The instant FRET reporter plasmids and recombinant FRET reporter cell lines are preferably quite stable and uniform in their expression of FRET reporter polypeptide, and the FRET signal (ratio) produced by PC and NC in different assays on different days preferably varies by less than about 10% to about 20%, preferably less than about 15%, e.g., PC from about 0.95 to about 1.05 and NC from about 1.27 to about 1.45.

Embodiments of the invention provide plasmid constructs encoding FRET reporter polypeptides having a variety of different linker polypeptides containing different substrates for different apoptosis proteases. In other embodiments, the instant methods provide for the preparation of a variety of different stable FRET reporter cell lines. Representative examples of presently preferred substrate domains which may be incorporated within the instant linker polypeptide, i.e., at the substrate domain “cassette” site, supra, are set forth in TABLE III, below.

TABLE III
Casette Site Substrate Domains

| | Linker Polypeptide |
|---|---|
| Apoptosis Protease | Substrate Domain |
| Caspase 1 | WEHD |
| Caspase 2 | DEHD |
| Caspase 3 | DEVD |
| Caspase 4 | (W/L)EHD |
| Caspase 5 | (W/L)EHD |
| Caspase 6 | VEHD |
| Caspase 7 | DEVD |
| Caspase 8 | LETD |
| Caspase 9 | LEHD |

Embodiments of the invention provide stable recombinant FRET reporter cell lines for identifying and selecting compounds capable of modulating apoptosis. Certain special requirements are placed on instant cells. In particular, the preferred instant cells are empirically determined to have all of the following properties: namely, (i) they are responsive to induction of apoptosis by extrinsic agents, preferably an apoptosis-inducing ligand, and most preferably anti-Fas antibody, which binds to a cell surface protein in the cells; (ii) they produce a measurable changes in FRET reporter signal within about 10 to about 24 hrs; (iii) the subject change in FRET reporter signal is accomplished with relatively low numbers of the cells in an assay, preferably about 40-100,000 cells in the assay, and most preferably, about 50-80,000 cells in the assay. Thus, on a per cell basis the preferred instant FRET reporter cells express levels of FRET reporter polypeptide that is about 5 μg to about 50 μg of the FRET reporter polypeptide per mg total cell protein, preferably about 20 μg/mg to about 40 μg/mg and most preferably about 30 μg/mg to about 40 μg/mg.

Embodiments of the invention provide plasmids encoding, and cells expressing, a FRET reporter polypeptide having a linker polypeptide optimized for binding apoptotic proteases. To preserve resonance transfer between the exciter and emitter fluorophores, i.e., a distance between the exciter and emitter that is within the Förster radius of the subject fluorophores, the instant linker polypeptide consists of a maximal length about 18 to about 40 amino acids, preferably about 18 to about 30, and most preferably, about 18 to about 25 amino acids. To maximize binding of the linker polypeptide to apoptotic proteases and hydrolysis of a peptide bond adjacent to, or at a position determined by, a substrate domain in a linker polypeptide, the presently preferred linker polypeptides consist of all of the following substituent domains linked in a serial array: namely, (i) an amino-terminal end-cap domain, located toward the amino terminus of the linker polypeptide and composed of about 3 to about 5 amino acids, wherein about 1 to about 3 of said amino acids are “end-cap” amino acids as defined supra; (ii) an acidic domain composed of about 3 to about 7 amino acids, wherein about 2 to about 5 of said amino acids are acidic amino acids as defined supra; (iii) a substrate domain composed of about 4 to about 7 amino acids selected for their ability to be cleaved by a protease induced during apoptosis, e.g., a caspase substrate site selected from TABLE III, above; (iv) a non-interfering domain (defined supra) composed of about 3 to about 5 amino acids; and, (v) a second end-cap domain near the carboxy terminus of the linker polypeptide that is composed of about 3 to about 7 amino acids, wherein about 1 to about 3 of said amino acids is again an end-cap amino acid as defined supra.

In yet other embodiments the invention provides nucleotide and amino acid sequences useful for constructing plasmids capable of expressing FRET reporters. The instant sequences are inserted, e.g., by ligation, site directed mutagenesis and the like, into a linker region consisting of about 75 nucleotides, located between and connected to nucleotide sequences encoding both an exciter and an emitter fluorophore. The instant exciter-linker-emitter nucleotide sequence is, in turn, introduced into any one of a variety of commonly available expression plasmids, as illustrated below. The instant exciter-linker-emitter plasmids may contain nucleotide sequences allowing their propagation in prokaryotic cells, but will always contain nucleotide sequences providing for expression of the instant FRET reporter polypeptide in a mammalian cell. At a minimum, DNA composing the instant plasmid consists of promoter and 5′ regulatory region elements operably linked to a FRET reporter nucleic acid in such manner that expression of the plasmid in a mammalian cell results in the expression of a FRET reporter polypeptide.

### EXAMPLE 1

### Feasibility of Cell-Based FRET Apoptosis Microtiter Well Assays

Experiments were initiated to investigate the feasibility of cell-based high-throughput microtiter assays for small-molecule inhibitors of apoptosis, and particularly assays useful for identifying intracellular targets involved in the processes of energy-dependent programmed cell death in neuronal cells. Considered key were determining: (i) whether FRET reporters were sensitive to non-specific proteolytic cleavage; (ii) what levels of FRET reporter expression might be required in a reporter cell line to detect apoptosis; (iii) whether the assay parameters required to detect a FRET reporter expressed in a cell line would be amenable to microtiter, and/or high throughput screening, assay formats; (iv) what assay parameters and formats might be required to detect intracellular cleavage of a FRET reporter, i.e., from within the large pool of available intracellular substrates; (v) what assay sensitivity would be required to detect small molecule inhibitors of apoptosis at non-toxic concentrations; and (vi) would the assays (as finally configured and validated) be sufficiently selective to notice differences between intracellular apoptotic and necrotic mechanisms of cell death.

Determining Operative Apoptotic Parameters in Host Cells: A variety of apoptosis inducer agents and cell lines have been described, however relatively little is still known about apoptotic responses triggered in a cell population, i.e., in microtiter culture, and in particular, in a high throughput screening type assay format. To achieve uniformity of signal in HTS it was considered important that after addition of an apoptosis inducer agent a relatively large proportion of the cells within any given cell population should commit to undergo apoptosis, and at relatively the same rates. To investigate the operational limitations several different available cell lines, apoptosis inducer agents and conditions were tested in microtiter plate formats, i.e. at about 1.5-2.0 ×10⁵ cells/cm in 24 well microtiter plates treated the following day with staurosporin, etoposide, anti-Fas antibody or doxorubicin. Assays evaluated included: assays for caspase-3 activity; SYTO 13 cell survival; Cell Death Detection ELISA^{PLUS}; Annexin V-FITC staining for phosphoinositide localization; DNA laddering (Qiagen BloodAmp genomic DNA isolation kit); TUNEL (Oncor ApopTag kit); microscopic analyses of propidium iodide-stained cells for an apoptotic phenotype, e.g., condensed nuclei and membrane blebbing; and assays for internucleosomal DNA fragmentation.

The results of these studies confirmed expectations, i.e., a considerable degree of variability existed in the results recorded in different assays with variability evident using different apoptosis inducers and different cell lines, and heterogeneity also evident at a microscopic level within a single population of cells. Thus, while apoptosis could be detected in microtiter format, it was not clear how assay variability and/or variability at a cell population level would affect a cell-based FRET apoptosis assay.

Determining Operative Parameters Required in a FRET reporter: A FRET reporter plasmid was constructed encoding a poly-His-tagged fusion protein containing the GFP mutants “Sapphire” and “Topaz” (Tsien et al. U.S. Pat. Ser. No. 5,625,048) linked by a polypeptide containing an apopain (caspase-3) substrate domain (i.e., DEVD). Briefly, the constructions utilized green fluorescent proteins (GFP), enhanced by random mutagenesis and selection, as derived according to methods substantially the same as those disclosed by Tsien et al. U.S. Patent Ser. No. 5,625,048 and Pollack et al. (9), both of which references are incorporated herein by reference. (GFP-encoding plasmids are also commercially available from Clontech.) Three such green enhanced mutant (GEM) GFP have been isolated (9), i.e., and referred to herein “Sapphire”, “Emerald” and “Topaz”. For use in the present studies, each was prepared as a coding sequence in the bacterial expression plasmid pRSET(A) (Invitrogen). The three plasmid constructs were used to transform (i) E. coli DH5α for preparation of bacterial and DNA stocks; and (ii) E. coli BL21(DE3) (Novagen) for production of FRET-reporter fusion protein. The GEM coding sequences were subcloned into pcDNA3 (Invitrogen) to produce mammalian expression plasmid vectors. Expression of the recombinant FRET-reporter fusion protein was evaluated by SDS-PAGE and fluorescence measurements.

Fusion proteins containing the FRET reporter were isolated and purified with the pRSET Xpress™ Kit (Invitrogen) according to the manufacturer's instructions. Briefly, a lysate was prepared from bacteria expressing the FRET reporter-fusion protein encoded by plasmid vector pRSET(A). The lysate was applied to a nickel resin column that binds to a poly-histidine tract in the fusion protein. The column was washed and the fusion protein was eluted with imidazole for fluorescent analysis and analysis by SDS-PAGE.

To estimate the limits of detection for the FRET reporter, different amounts of the Emerald fusion protein were added to a microtiter plate and assayed in a Cytofluor II plate reader (PerSeptive Biosystems) at concentrations of 100, 50, 25, 10, 5 and 1 μg/ml in PBS (excitation 485 nm, emission 530 nm). The Emerald fluorescence signal ranged from nearly 40-fold greater than background at 100 μg/ml protein to seven-fold greater than background at the 1 μg/ml concentration. Since each mictotiter assay well contained about 100 μl of the fusion protein solution, the calculated required expression levels of FRET reporter polypeptide to be detectable at 7-fold greater than background in a culture of FRET reporter cells was predicted to be about 100 ng. Given that a maximum of about 100,000 to 200,000 cells are commonly cultured in a single microtiter well, these findings suggested that a high level of expression of FRET reporter polypeptide might be needed to achieve results in such a cell-based FRET reporter assay, e.g., expression at a level >1% of total cell protein. While this level of expression is possible, expression of foreign proteins in mammalian cells at these levels can be toxic. Therefore, efforts were made to increase the sensitivity of the assay and to establish conditions under which lower levels of expression could be assayed without toxicity.

For FRET reporter proteins to detect apoptotic proteases they must necessarily not be subject to cleavage by other proteases expressed in normal or apoptotic cells e.g., serine proteases. For the FRET reporter proteins to be useful as substrates for apoptotic proteases also requires that cleavage occur at a specific substrate site and not at some other non-specific site in a fluorophore. The possibility existed that reporters containing Emerald, Sapphire or Topaz GFPs might be susceptible to non-specific cleavage by proteases (6). Experiments were therefore performed to evaluate susceptibility of Emerald GFP, i.e., in a fusion protein, to proteolytic cleavage by trypsin (i.e., a serine protease) and apopain (i.e., a cysteinyl protease). For these experiments 10 μg aliquots of Emerald fusion protein in PBS were incubated with trypsin (1 μg/ml) or apopain (3.3, 1.7 or 1 μg/ml) and assayed for fluorescence at five-minute intervals over the course of one hour. No decrease in emission relative to an untreated control was observed during this time. Thus, under these conditions of treatment the Emerald fluorophore was resistant to cleavage by trypsin and apopain. Since the amino acid sequences of Sapphire and Topaz differ only slightly from that of Emerald, these results suggested that serine and cysteinyl proteases might not be able to mediate cleavage within the fluorophore portions of an Emerald-Topaz or Sapphire-Topaz FRET reporter. However, ultimately this could only be determined by expressing these FRET reporters in mammalian cells.

Construction of Initial Test FRET Reporters and Determination of Operative Properties Required in a Linker Polypeptide: As a first step, nucleotide sequences encoding the GEMs “Sapphire” and “Topaz” were used for construction of two FRET test reporters, i.e., Sapphire-Linker-T-Topaz (STT; containing a trypsin substrate domain) and Sapphire-Linker-A-Topaz (SAT; containing an apopain/caspase-3 substrate domain; see the Materials and Methods section, below). In STT and SAT the two Sapphire and Topaz fluorophore coding sequences were joined together through a nucleotide sequence encoding a linker polypeptide consisting of 25 amino acid residues. The linkers were modified from a disclosed trypsin-cleavable linker BFP-GFP construct (7) as follows: namely, (i) to facilitate assembly of the construct, mutagenesis was used to introduce a restriction site into the 3′ terminus of SAT- and STT-linker nucleotide sequences; (ii) a substrate domain specific for apopain/caspase-3 was produced by mutation of two codons within the SAT linker; and, (iii) an enterokinase substrate site within the disclosed construct to produce the disclosed linker was eliminated by mutation in SAT. The resulting modifications produced a “cassette” linker region bounded by restriction sites. The trypsin substrate site was retained in the SAT linker so that trypsin could be used as a comparative control. A comparison of the sequence of the STT and SAT linkers with the sequence of the disclosed BFP-GFP linker (7) appears in TABLE IV.

TABLE IV
Amino Acid Sequences of FRET reporter Linker Polypeptides*

| | | | |
|---|---|---|---|
| BFP-GFP | [S65C]- | S S M T G G Q Q M G R D L Y D D D D K D P P A E F | -[BFP] |
| STT | [Sapphire]- | S S M T G G Q Q M G R D L Y D D D D K D P P A E A | -[Topaz] |
| SAT | [Sapphire]- | S S M H G G Q Q M G R D E V D G D D K D P P A A A | -[Topaz] |

*Potential protease substrate sites: R (trypsin), DEVD (apopain), DDDDK (enterokinase).

Determining Operative Parameters for Detecting FRET Changes: Purified recombinant FRET reporter proteins were used in a 24-well microtiter plate assay to evaluate changes in FRET signal produced by protease treatment.

STT and SAT FRET reporter fusion proteins were expressed in cultures of E. coli BL21(DE3) transformed with pRSET(A)-SAT or pRSET(A)-STT and induced with 0.5 mM IPTG for 4 hr. Approximately 1 mg of SAT and 0.4 mg of STT fusion proteins were partially purified using Ni—NTA Superflow nickel resin (Qiagen) and FPLC (see Methods). The STT protein was fluorescent but the SAT protein was not (excitation 395 nm, emission 530 nm).

To evaluate the susceptibility of these SAT and STT fusion proteins to proteases, aliquots of the purified proteins were treated with trypsin (0.25-0.5 mg/ml, 20 min) and analyzed by western blot using a polyclonal anti-GFP antibody (Clontech). Several bands were detected between ˜30-60 kDa in the non-treated (negative control) STT lane, but only ˜30-40 kDa bands were present in trypsin-treated STT lanes. These results were consistent with the predicted sizes of ˜60 kDa for the full-length reporter and ˜30 kDa for the individual Sapphire and Topaz constituents, i.e., released by spacer cleavage. SAT protein was not detectable, suggesting that this particular bacterial system could not produce a functional SAT reporter.

The fluorescent bacterial STT reporter protein, above, and a Sapphire fusion protein control were treated with trypsin and analyzed to detect cleavage by both western blot analysis, i.e., using a monoclonal GFP antibody (Clontech), and fluorescence measurements in the microplate assay described above. Aliquots of purified reporter protein were assayed for fluorescence in a 24 microtiter plate format using a Cytofluor II plate reader (PerSeptive Biosystems) (6 reads/well, gain 70). Aliquots of purified reporter protein were excited at 395 nm and fluorescence was measured with two narrow bandpass filters: 495/10, which detects Sapphire but not Topaz emissions, and 530/10, which detects both Sapphire and Topaz emissions (excitation filter from PerSeptive Biosystems, emission filters from Chroma Technologies). Calculation of the background-corrected 530:495 ratio allowed Sapphire background fluorescence to be mathematically filtered out, i.e., a decrease in the 530:495 ratio indicated a decrease in FRET.

In control samples, the major species detected by western blot analysis was approximately 60 kDa, i.e., consistent with the predicted size of a full-length STT fusion protein, although smaller species, i.e., in the 30-40 kDa size range, were also detected suggesting either cleavage or incomplete synthesis of the STT reporter protein. Following a 30 minute trypsin treatment, the only detectable bands were in the 30-40 kDa range, i.e., the size predicted for cleavage products. These findings suggested that cleavage of the 60 kDa reporter was essentially complete within 30 min. Fluorescence studies further revealed about a 15% decrease in FRET signal, i.e., as expressed by the 530:495 ratio, within the first 15 min with no further decrease over the next 30 min, suggesting that, under these conditions, reporter cleavage was complete within 15 minutes. The 530:495 ratio of untreated STT controls remained relatively constant over the entire time course, indicating the intrinsic stability of the STT reporter protein. Thus, the control experiments with purified protein indicated that the maximal FRET signal change resulting from complete trypsin cleavage of linkers (similar to those disclosed in the prior art) elicited only about a 15% change in FRET signal. These findings suggested that an improved reporter design and/or detection method might be needed to effectively measure apoptotic changes in cells.

### EXAMPLE 2

### HEK293 FRET-Reporter Cell-Based Apoptosis Test Systems

A test system was constructed to evaluate SAT reporter expression and feasibility of detecting FRET signal changes in mammalian cells in microtiter assays. The human embryonic kidney-derived HEK293 cell line was chosen as a host because of its relatively high transfection efficiency, rapid growth, easy maintenance and suitability for microplate assay formats. In preliminary experiments in which HEK293 cells were transfected with pcDNA3-Emerald, transformed cells were fluorescent and there was no loss of fluorescence, or obvious detrimental effects on cell morphology or growth, in a selected pool of cells (i.e., mixed populations of cells) over the course of one month. It was also possible to isolate individual stable colonies from within these selected pools of transfected HEK293 cells, in this case, clones of cells having FRET reporter DNA integrated into their genome, derived from cells being found to be present in mixed populations of selected cells after about 2-4 weeks of selection. Optimal conditions for transfection of HEK 293 cells with SAT-reporter plasmid were determined. Cells transfected using LIPOFECTAMINE (Life Technologies) and selected in G418 (Life Technologies) expressed full-length SAT reporter protein as determined by western analysis of transformed HEK293 cells after two weeks of in vitro selection. To determine whether caspase-3 activity could be induced, the SAT-reporter-transformed HEK293 cultures were treated with 100-200 μM etoposide or 0.5-10 μM staurosporin for 4, 5, 8 or 24 hrs. and assayed for caspase-3 (Materials and Methods, below). After 8 hrs. treatment, caspase-3 activity was increased six- to 10-fold over background in 5 μM staurosporin-induced HEK293 cells, but no change was observed in etoposide-induced cells. Under these conditions of treatment with staurosporin only about half of the SAT-reporter-transformed HEK293 cells in the population were apoptotic at 4 hrs. as determined using the Annexin V assay (Materials and Methods). These combined findings suggested limitations in the use of short-term transiently transfected cells, the possible advantages of using long-term stable (i.e., 2-4 week cultures), but also the possible need for both optimization of reporters, and selection of certain clonal populations of cells in which apoptosis could be induced at a high level. Absent these conditions, it was considered unlikely that apoptosis-related FRET signal changes would be of sufficient magnitude to be detectable in a microtiter plate HTS assay.

Appropriate cleavage of the FRET reporter by proteases requires correct folding and accessibility of the linker to the protease. To determine whether SAT polypeptide expressed in SAT-transformed HEK293 cells was sensitive to apoptotic proteases, selected pools of transiently transfected HEK293 cells expressing either the full SAT reporter protein or just the Sapphire exciter fluorophore, were extracted and the cell extracts subject to trypsin treatment. Possible cleavage of SAT-reporter and/or Sapphire GFP protein in the cell lysates was determined by measuring changes in FRET fluorescence and western blot analysis. The SAT reporter protein was sensitive to trypsin, i.e., FRET signal decreased and western blot analysis showed cleavage, and the Sapphire GFP expressed in control cells was not cleaved. These findings with cell extracts suggested that FRET reporter synthesized by cells was sensitive to cleavage by proteases at the desired site in the linker region, and not elsewhere.

HEK293 FRET Reporter Cell Lines: In an attempt to overcome some of the difficulties encountered in use of pools of selected transfected cells (i.e., after 2-4 weeks selection, above), HEK293 cells were transfected (i.e., 6 μg pcDNA3-SAT plasmid DNA; LIPOFECTAMINE™) and selected in G418 (500 μg/ml) for 2 weeks, at which time 12 individual fluorescent colonies, i.e.,. F1-1 to F1-12, were isolated and expression of reporter fluorophore was confirmed by western blot analysis. (A pool of transfected cells was also maintained under selection.) Of the 12 parental cultures, F1-1 and F1-2 displayed the strongest fluorescent signals. Expression of SAT reporter fluorophore in 12 selected cell lines was confirmed by western blot analysis (i.e., using monoclonal GFP antibody) of cell lysates. Each of the cell lysates showed strong bands at ˜60 kDa and ˜30 kDa, i.e., the expected sizes for full-length SAT trypsin-cleavable reporter protein and for the single GFP fluorophore molecules, respectively. In addition, cells in the selected F1 pool expressed an apparent greater percentage of full-length (60 kDa) SAT reporter than was expressed in a culture of transiently transfected HEK293 cells, i.e., ˜90% full length vs. ˜50%, respectively. F1-1 and F1-2 were subcloned by limiting dilution and reporter fluorescence assayed in four of the subclones was shown to be stable through at least 13 passages. Subclone F1-1-42 (ATCC No. PTA-83) was selected for initial testing of reporter cleavage and apoptosis-induced FRET changes. The fluorescence signal produced in F1-1-42 cells was found to be stable through at least 21 passages, i.e., conducted over 10 ½ weeks.

Detecting Intracellular Changes in a FRET Reporter in Cells Undergoing Apoptosis: Having established that SAT reporter protein synthesized in HEK293 cells could be cleaved ex vivo, i.e., in cell lysates by trypsin (above), an initial in vivo intracellular assay was conducted using the selected F1-1-42 clonal cell line and staurosporin to induce apoptosis. Objective evidence was obtained in these experiments that the addition of an extracellular apoptosis inducer agent to the cells, i.e., staurosporine, resulted in intracellular events that decreased the signal produced by a FRET reporter, i.e., the SAT reporter, and the observed changes in FRET signal were correlated with cleavage of the SAT reporter, i.e., by western blot analysis. In these experiments the F1-1-42 cells were plated at a density of 4×10⁵ cells/0.5 ml in each well of a 24-well microtiter plate. After overnight incubation, apoptosis was induced by adding staurosporin to a final concentration of 5 μM, and using vehicle (0.25% DMSO) as a negative control. Assays for caspase-3 were used to confirm that apoptosis was induced. Final analysis of the data revealed caspase-3 activation, cleavage of the SAT reporter by western analysis and a concomitant decrease in FRET signal. Subsequent testing determined that significant changes in FRET signal were first detectable at about 8 hours after addition of staurosporin and were maximal by 24 hrs post-induction. These results demonstrated for the first time that changes in FRET reporter proteins could be detected in living cultures of cells following induction of apoptosis, and that cleavage of the FRET reporter could be monitored by periodically measuring changes in FRET signal in a microtiter assay where cells were undergoing programmed cell death.

To further confirm that the staurosporin-induced change in FRET signal was a result of caspase (apoptotic protease) cleavage of the SAT reporter and not some other proteolytic event, attempts were made to block cleavage using a cell-permeable caspase-3 specific inhibitor, i.e., Z-DEVD-fmk (Enzyme Systems Products). When Z-DEVD-fmk (100 μM) was added to cultures of F1-1-42 cells 30 min prior to addition of staurosporin, the inhibitor reduced morphologic signs of apoptosis (i.e., less rounding of cells) and there was markedly less cleavage of SAT reporter in cell extracts (monitored by western blot) and a marked reduction in the FRET signal change. Vehicle-treated control cultures did not show these changes.

In general, HEK293 cells were helpful in establishing that a GFP-based FRET reporter could be expressed stably in a mammalian cell line and at levels sufficient to detect FRET signal changes in living cells after induction of apoptosis. However, while human embryonic kidney cells were thought to be relatively useful in screening assays for identifying inhibitors of apoptosis in cells derived from embyonic mesenchymal tissues, they were believed not to be a good system for modeling apoptotic changes in differentiated cells of the central nervous system. Experiments were therefore initiated to evaluate expression of FRET reporters in CNS-derived cells in hopes that development of FRET reporter cell lines from those sources might be useful in screening assays designed to select and identify compounds useful in treating neurological diseases.

### EXAMPLE 3

### CNS Host-Cell FRET-Reporter Apoptosis Assay System

CNS FRET-Reporter Host Cells: Criteria considered requisite for selecting an acceptable CNS-like host cell line useful in high throughput screening included: (1) relative hardiness in the presence of potential toxic test agents; (2) rapid growth and undemanding culture requirements (i.e., media, growth factors and substrate); (3) an acceptable >5% transfection efficiency (i.e., >5% of cells compared with about >50%-80% needed for transiently transfected cells); (4) the ability, on the part of the cells, to synthesize, process and fold a functional FRET reporter protein; (5) the ability of the cells to express the FRET reporter protein at levels effective to produce FRET signal changes detectable in apoptotic cultures in a micotiter assay format; (6) induction of apoptosis in more than 50% of the cells in an assay well; (7) induction of apoptosis in the cells by at least one, and preferably two, apoptosis inducer agents; and (8) adherence, i.e., in apoptotic cell cultures preferably 80% of the cells remain adherent at 8-18 hrs. after induction of apoptosis.

Three CNS derived cell lines, IMR-32, NB41A3 and Neuro2A, were initially evaluated for growth and transfection characteristics, inducibility of apoptosis in a large percentage of cells, and a inducible apoptosis protease activity. From among the three cell lines, only the IMR-32 cell line showed detectable levels of caspase-3 activity by western analysis and therefore it was the only one initially selected for further examination. The SYTO 13 cell survival assay and Cell Death Detection ELISA^{PLUS} (Materials and Methods) were used to optimize conditions for induction of apoptosis. IMR-32 cells were treated with Actinomycin D (ActD), cycloheximide (CHX), etoposide or staurosporin for 4-8 hrs. In these experiments programmed cell death induced by ActD, CHX and etoposide, i.e., as measured by Cell Death Detection at 4-8hrs., was only about 30% with ActD and about 50% with CHX and etoposide. However, DNA fragmentation as determined by ELISA showed dose-dependent changes only for etoposide, and not for CHX or ActD, i.e., suggesting non-specific toxic effects of the latter two agents in this test cell system. To confirm induction of apoptosis by etoposide, IMR-32 cells were treated with 50 μM etoposide and monitored over 24 hours for cell survival, internucleosomal DNA fragmentation and morphological changes indicative of apoptosis. The SYTO 13 assay showed 55% of cells surviving at 8 hr and just 30% surviving at 24 hrs. In agreement, DNA fragmentation reached a maximal value at 8 hrs. Cells stained with SYTO 13 and propidium iodide following etoposide-treatment and examined microscopically showed that about 50% of the cells attached to the plates at 8 hrs. contained condensed nuclei and membrane blebbing but none were obviously dead; even at 24 hrs. where about 75% of the attached cells showed apoptotic morphology; only about 15% were clearly dead. The observed discrepancy between results recorded in DNA cleavage ELISA assays and Cell Death Detection assays were attributable to loss of adherence in dead and dying cells. The non-adherent cells thus became detached from plates during the course of the assay resulting in a variable and uncontrolled loss of signal. Thus, under these conditions of assay floating cells seemed to contained the majority of the dead and dying cells. Subsequent experiments using Annexin V and caspase-3 assays confirmed apoptotic events in IMR-32 cells treated with etoposide (i.e., 50-100 μM), i.e., by Annexin V staining (below) and about a 10-fold induction of caspase-3-like activity. In contrast, Doxorubicin (5 μM) was also tested, but it did not produce any increase in caspase-3 activity.

Based on these combined findings, the IMR-32 appeared to be a possible host cell for a cell-based FRET assay, but subsequently the following significant drawbacks were observed: namely, (1) an unacceptable level of assay variability, i.e., determined to be due to a constantly changing proportion of cells floating at different distances from the fluorescence detector; and (2) an unacceptable loss of cells and FRET signal, when cells were subject to washing, i.e. to remove media and test compounds which might contribute to background auto-fluorescence. Thus, IMR-32 apparently exhibited decreased cellular adherence to substrates when apoptosis was induced. Nonetheless, since no other test cells had been shown (at the time) to be susceptible to apoptosis induction, attempts were made to stably express the SAT reporter in these cells.

IMR-32 Host Cells: IMR-32 cells were transfected with 6 μg pcDNA3-SAT using LIPOFECTAMINE™ (Life Technologies) according to standard procedures, but colonies lifted off the plate during selection (G418, 500 μg/ml). Transfection conditions were modified, i.e., using plates coated with Matrigel (Collaborative Biomedical Products) to improve cell adherence and reducing the selection pressure to 100 μg/ml G418, but these changes still resulted in no improvement: i.e., no colonies survived. Next, a study was conducted of possible transfection and selection conditions that might be acceptable for use with the IMR-32 cells. Six different expression vectors and three antibiotics were evaluated in this study: namely, pcDNA3 (Invitrogen), pZeoCMV, pZeoSV (Invitrogen), pHygCMV, pHook-3 (Invitrogen) and pMClneoPolyA (Stratagene); and, G418, Zeocin, or hygromycin B. However, since only the pHygCMV/hygromycinB combination yielded healthy colonies, the SAT reporter coding sequence was subcloned from pcDNA3-SAT into pHygCMV. When the pHygCMV-SAT expression construct was transfected into IMR-32 cells, (i.e., using the standard LIPOFECTAMINE protocol), few cells survived selection with hygromycin (50 μg/ml) and none were fluorescent or capable of sustained growth. Thus, after significant expenditure of effort, the IMR-32 cells were finally considered to be unsuitable and alternate CNS-like host cell lines were explored.

U-138 Glioblastoma Host Cells: The U-138 MG human glioblastoma cell line (ATCC #HTB 16) was evaluated a potential host. This cell line was found to be hardy, strongly adherent and relatively easily cultured and transfected. The cells were maintained in 2 MM media (DMEM with L-glutamine, 6% fetal bovine serum, 100 U/ml penicillin, 100 μg/ml streptomycin) and passaged 1:5 or 1:10 every 3-4 days. Experiments were conducted to test inducibility of apoptosis with 30-300 μM etoposide or 1.5-15.0 μM staurosporin, in this case, as measured by determining the levels of caspase-3 activity at 8 hrs. Caspase-3 activity was found to be inducible with staurosporin but not with etoposide. Since 5-15 μM staurosporin induced similar levels of caspase activity, 5 μM staurosporin was chosen for subsequent experiments. In kinetic experiments, caspase-3 activity and inter-nucleosomal DNA fragmentation assays (Materials and Methods) were used to monitor the progress of apoptosis after addition of 5 μM staurosporin. Both measures of apoptosis were minimal at 4 hrs., intermediate but significantly different by 8 hrs., and maximally altered at 16-24 hrs. after induction, thus confirming proper and useful kinetics of apoptosis induction in the U-138 cells.

Comparative studies of three different transfection protocols revealed that a modified calcium phosphate method (Materials and Methods) yielded significantly better transfection efficiencies than either of LIPOFECTAMINE (Life Technologies) or SuperFect (Qiagen) with U-138 cells. Stable expression of the SAT reporter in U-138 cells was investigated by transfecting cultures with pcDNA3VH-SAT using calcium phosphate (Materials and Methods), and then selecting in medium containing G418 (400 μ/ml). Fourteen parental colonies were expanded and tested, and about 50-95% of the cells within each of these parental populations was fluorescent, i.e., suggesting a relatively good correspondence between transfection and selection of the selectable marker and expression of the FRET reporter. To investigate functional expression of the FRET reporter in the transfected U-138 cells, experiments were conducted and it was determined that staurosporin induced changes in FRET signal and the changes in signal were correlated with cleavage of the FRET reporter in western blot analysis. The parental culture showing the strongest fluorescence and best growth characteristics was subcloned. Unexpectedly, the ability of the subclones to produce a staurosporin-inducible change in FRET signal was not stable, i.e., disappearing at passages 9, 11 and 13, but then reappearing at passage 16. While it was speculated that this phenomena may have resulted from some difference in expression and/or regulation of apoptosis or FRET reporter in sub-cultures of cells, these properties were not considered desirable and these SAT-transfected U-138 cell lines were discontinued in hopes that an improved reporter construct might solve the problem of irreproducibility.

### EXAMPLE 4

### Design and Construction of Improved FRET Reporters

The SAT reporter protein, as expressed from pcDNA3-SAT, should theoretically comprise a ˜60 kDa protein made up of GFP mutants Sapphire and Topaz (Aurora Biosciences) linked together through a linker polypeptide containing both a potential apopain/caspase-3 site (i.e., DEVD) and trypsin cleavage sites (i.e., K and R). However, in experiments described above in EXAMPLES 2 and 3, western blot analysis of transiently transfected cells showed a high proportion of ˜30 kDa species, i.e., suggesting either intracellular non-specific cleavage of FRET reporter or incomplete synthesis with expression of single non-linked GFP fluorophores. At least three different alternatives were considered: namely, (1) problems relating to expression of full-length reporter; (2) problems intrinsic within the mutant GFP polypeptides (Sapphire and/or Topaz); or, (3) problems resulting from intracellular cleavage. To distinguish between these different possibilities, experiments were conducted to evaluate expression of SAT reporter in the HEK293 cells. As a first step, the SAT coding sequence was inserted into mammalian expression vector pcDNA3.1NV5-His (Invitrogen) resulting in pcDNA3VH-SAT. However, when transfected into HEK293 cells, the signal produced by either the new pcDNA3VH-SAT, or the old pcDNA3-SAT were similar. Since no improvement was observed by changing the expression vector, the results suggested that expression may not be the problem. To determine whether the problem lay in intracellular cleavage of the SAT linker polypeptide by intracellular proteases, attempts were made to construct reporters with improved linker regions, i.e., lacking potential serine protease sites and optimized for recognition by cysteinyl apoptotic proteases.

Optimization of a new linker polypeptide was initiated by comparing the sequences of different known apoptosis substrates (TABLE V).

TABLE V
Amino Acid Sequence Analysis: Design of
the gA and nA Test Linker Polypeptides
Caspase-3 Cleavage Site and Neighboring
Residues in Six Endogenous Substrates

| | |
|---|---|
| PARP | G V K S E G K R K G D E V D G V D E V A K K K S K |
| DNA-PK | F G K K R L G L P G D E V D N K V K G A A G R T D |
| U1-70K | D D G P P G E L G P D G P D G P E E K G R D R D R |
| SREBP | S P L L D D A K V K D E P D S P P V A L G M V D R |
| D4-GDI | P E P H V E E D D D D E L D S K L N Y K P P P Q K |
| huntingtin | C D L T S S A T D G D E E D I L S H S S S Q V S A |

Composition of the Above Sequences

| | | |
|---|---|---|
| Charged: | ˜40% | Most Common (descending order of frequency): DGEKPSVL |
| Acidic | ˜30% | Least Common(ascending order of frequency): WCIMFYH |
| Basic: | ˜10% | Profile: Very hydrophilic and negatively charged |
| Polar: | ˜15% | `````````(especially centrally); positive charges |
| Hydrophobic: | ˜20% | `````````at one/both ends. |

Test Linker Sequences

| | |
|---|---|
| gA | S P L H S G E D D G D E V D G V E N V H P P A T A |
| nA | S P L H S G E D D G D E V A G V E N V H P P A T A |

PARP: poly(ADP-ribose) polymerase; DNA-PK: 460-kDa subunit of DNA-dependent protein kinase; U1-70K: 70-kDa subunit of U1 small ribonucleoprotein; SREBP: sterol regulatory element binding protein; D4-GDI: D4 GDP dissociation inhibitor; huntingtin: HD gene product.

A caspase-3 cleavage site domain consisting of 25 amino acid residues was evaluated in each of the 6 substrates (TABLE V). Overall, within these 25 amino acids 44% were charged (i.e., RKHYCDE), 27% were acidic (i.e., D, E), 11% were basic (e.g., K), 15% were polar (i.e., NCQSTY), 20% were hydrophobic (i.e., AILFWV); the most common residues were D (17%), G (12%), E, K and P (10%), S or V (8%) and L (7%); and, the least common residues were W (10%), C, I, M, F, or Y (0.7%) or H (1.4%). The net overall properties of the 25 amino acid region were hydrophilic, negatively charged, i.e., especially in the center between positions 7 and 12, with one or both ends frequently positively charged. Common features within this domain were investigated further by constructing test plasmids capable of encoding Sapphire and Topaz connected through two different linker polypeptides. The first was a “generic apopain” (gA) linker containing a caspase-3 cleavage site, and the second was a negative control “not apopain” (nA) linker. The new Sapphire-linker-Topaz reporter (SgAT) and control (SnAT) constructs contained either a potential caspase-3 cleavage site, i.e., DEVD (SgAT), or, as a specificity control, the sequence DEVA (SnAT). Both of the linkers also lacked potential cleavage sites for trypsin or other serine proteases (i.e., K and R). The SgAT and SnAT nucleotide coding sequences were assembled 5′ to a C-terminal His₆ tag in vector pcDNA3.1/V5-His (Invitrogen). Synthetic PCR and standard cloning techniques were used to generate constructs pcDNA3VH-SgAT and pcDNA3VH-SnAT in vector pcDNA3.1/V5-His.

### EXAMPLE 5

### Improved FRET Reporter Cell Lines

Transfection of pcDNA3VH-SgAT and pcDNA3VH-SnAT into U-138 cells (using methods described above) resulted initially in few colonies and FRET signal was lost in many clones during expansion, however, from among the various subclones the F9-1-13 (pcDNA3VH-SgAT transfected; ATCC No. PTA-84) and F10-24-2 (pcDNA3-SnAT transfected) subclones were eventually expanded and shown to express stable FRET signals. As desired, the FRET signal expressed in F9-1-13 decreased in response to both staurosporin and anti-Fas antibody treatment of cells, while the FRET signal expressed by the F10-24-2 control cells, (i.e., lacking a caspase substrate domain), was unaffected following staurosporin or anti-Fas antibody treatment. Kinetics of changes in FRET signal and caspase-3 activity were appropriate for induction of apoptosis, i.e., changes in FRET signal were significantly different from vehicle-treated induced cultures at 8 hrs. and the differences were maximally different at about 16-24 hrs. Western blot analysis showed only full-length (60 kDa) FRET reporter protein in lysates from untreated F9-1-13 and F10-24-2 cells, indicating that the cells were apparently successfully producing full-length SgAT and SnAT proteins without truncation and/or cleavage. Following staurosporin induction F9-1-13 reporter cells showed strong bands in both the 60 kDa and 30-40 kDa size ranges, indicating significant (but not complete) cleavage of reporter, whereas the F10-24-2 (negative control) cells showed no visible difference before and after induction of apoptosis with staurosporin. The inducibility of a FRET signal change in F9-1-13 cells in response to staurosporin treatment was stable over at least 50 passage (i.e., about 25 weeks), suggesting that the difficulties initially encountered with stability and apparent reporter cleavage (i.e., in EXAMPLE 3, above) were likely due to the choice of linker region sequence. F9-1-13 therefore appeared suitable for microtiter assays, and perhaps use in HTS assays for screening to identify and select modulators of apoptosis.

### EXAMPLE 6

### Establishing and Validating a Cell-Based FRET Reporter Microtiter Assay for Selecting and Identifying Modulators of Apoptosis:

Studies were initiated to adapt the experimental cell-based FRET assay of EXAMPLE 5 to a format suitable for use in high-throughput screening. First, the assay was scaled down from a 24-well to a 96-well plate format. Cells from three different F9 parental cultures were plated in a black-sided 96-well plates (Costar #3904) at a density of 32,000 cells/well (˜1×10⁵ cells/cm²); allowed to adhere overnight; and then the media was replaced with either 100 μl fresh media containing inducer (5 μM staurosporin in 0.25% DMSO vehicle) or 100 μl containing only vehicle (0.25% DMSO). After an overnight incubation, fluorescence measurements were made to evaluate FRET signal changes. All three of the parental cell cultures showed a significant (P<0.05) reduction in the FRET signal compared to the vehicle control, but the relative change in fluorescence units (RFU; 495/10 filter) were quite low (i.e., ˜20-60 RFU greater than background). The assay therefore appeared to be adaptable to a 96-well format but it was not initially clear how signal might be improved to increase the signal-to-background ratio.

Optimization: Parameters evaluated and optimized included the following: namely, number of cells per assay, number of wells per assay, time, type and amount of apoptosis inducer agent, duration of fluorescence measurements, type of fluorescence measuring device, inter-well/intra-plate variability, inter-plate variability, culture conditions and the like.

A 96-well FRET assay was evaluated for inter-assay well variability and inter-plate variability. Possible signal drift during measurement of the FRET fluorescence signal was evaluated using one of two different robotics-compatible rapid fluorescence plate readers (SpeedReader or Cytofluor II). After plating at an initial density of about 60,000 cells/well, and overnight culture F9-1-13 cells were treated with 5 μM staurosporin, or vehicle. After an overnight incubation in staurosporin (i.e., 16-24 hrs.), the plates were either washed, or not washed, and then assayed repeatedly over the next 1.5 hours using either of the plate readers. At an initial plating density of 60,000 cells/well in duplicate plates F9-1-13 cells were found to produce measurable changes in FRET signal (using either fluorescence reader) within about 4 hours when treated with 5 μM staurosporine, i.e. as compared with vehicle-treated wells. However, using the same assay conditions but with anti-Fas antibody as an inducer, different results were recorded. First, the SpeedReader showed no significant differences between experimental and vehicle control cultures; and, large inter-well variability was observed within plates that tended to obscure apoptosis-induced changes in the FRET signal. In contrast, measurements made with the Cytofluor II revealed statistically significant differences between treated and control group mean RFU (P<0.001); relatively small standard deviations of the mean for the values recorded within each treatment group, (i.e., n=4 wells); relatively little inter-well variability within a plate or variability between different wells in different replicate plates. The basis for the observed differences in measuring devices is not clear, but may relate to the design of the light source and/or the sensitivity of the detection circuitry in the two different respective fluorescence readers as well as possible different means for optically or digitally subtracting background incident radiation.

In multiple assays, and in HTS assay formats, cells of the F9-1-13 stable FRET reporter cell line have been found to produce reliable and consistent results over a relatively broad window of time when a plate reader, i.e., when an appropriate optical fluorescence reader is used. The Cytofluor II was selected as the preferred plate reader for a high-throughput FRET assay.

Assay Confirmation: The following experiment was performed to validate the ability of the 96-well FRET assay to detect inhibitors of apoptosis: namely, F9-1-13 cells (n=5 wells) were treated with 50-100 μM Z-VAD-fmk (a broad spectrum caspase inhibitor) or vehicle (n=55) in randomized wells of a 96-well plate. All of these wells, and six additional positive control wells (i.e., lacking the inhibitor), were treated with 5 μM staurosporine in 0.25% DMSO vehicle. Six negative control wells were treated with vehicle only (0.25% DMSO). After overnight incubation, FRET signal changes were evaluated as described above. The FRET signals within the different respective groups were tightly clustered, and the Z-VAD-fmk group mean was significantly (P<0.001) higher than the mean FRET signal recorded in the staurosporine treatment group, i.e., ˜65% greater indicative of Z-VAD-fmk inhibition of the apoptosis-induced decrease in FRET signal. These results confirmed that the 96-well assay format using F9-1-13 cells was capable of measuring intracellular changes triggered during apoptosis.

Optimization for High-Throughput Screening: Subsequent experiments evaluating the effect of various factors on FRET signal and background were performed to optimize the assay for high-throughput screening. Increasing the cell density to 1.5×10⁵ or 2.2×10⁵ cells/cm² (50,000 or 75,000 cells/well) increased the RFU detected per well, with no apparent improvement in the apoptosis-induced FRET signal ratio (as defined supra).

Three alternatives to staurosporine induction were evaluated: serum withdrawal; c2-ceramide treatment; and induction via engagement of the CD95/Fas receptor with anti-Fas antibody. Only a small reduction in the FRET signal and increase in caspase-3 activity was observed following 16-24 hours treatment with 75 μM c2-ceramide (Sigma). No effects of serum deprivation on FRET or caspase-3 activity were observed at 16-24 hours. In contrast, anti-human Fas antibody (i.e., 125-2000 ng/ml; Upstate Biotechnology) showed a clear dose-dependent change in FRET, reporter cleavage (i.e., by western analysis) and caspase-3 activity in F9-1-13 cells. In these experiments, the change in FRET induced by 1 μ/ml anti-Fas antibody was comparable to that induced by 5 μM staurosporin, and unlike staurosporin, most of the cells still appeared to be firmly attached to the plate after 16-24 hrs. of anti-Fas treatment. Subsequent experiments indicated that 300-500 ng/ml anti-Fas was sufficient to induce a substantial and consistent reduction in the FRET signal after 16-24 hours, and that gentle washing with 1×PBS was well-tolerated with little or no loss of cells. To increase the proportion of adherent cells, poly-D-lysine and 0.1% gelatin were evaluated in anti-Fas antibody treated F9-1-13 cells. Plates coated with 0.1% gelatin supported cells treated overnight with anti-Fas antibody showed little or no loss of cells when washed with 1×PBS, whereas cells treated with 5 μM staurosporin showed significantly reduced adherence under all test conditions (i.e., 50-100% of the cells were lost after the wash). Anti-Fas antibody was therefore chosen as a preferred inducer of apoptosis for high-throughput screening.

Using anti-Fas antibody as an apoptosis inducer, cell densities in the range of 10,000-60,000 cells/well were evaluated in the FRET assay. RFU increased proportionately with increasing cell density, and 20,000-60,000 cells/well (0.7×10⁵-1.9×10⁵ cells/cm²) yielded acceptable apoptosis-induced changes in FRET signal. For HTS, 60,000 cells/well was selected.

### EXAMPLE 7

### High-Throughput Screening Assays

Proteases induced during apoptosis may be distantly related to earlier cellular events triggered by apoptosis inducers. Theoretically, interfering with any cellular component of an induced apoptosis pathway, at or following apoptosis induction and prior to caspase-3 activation, will inhibit the apoptosis-induced change in FRET signal in F9-1-13 cells. For example, apoptosis modulators identified using F9-1-13 cells may include inhibitors of signal transduction events, inhibitors of cofactor-procaspase complexes (i.e., required for activation of caspase-8 or -9), and modulators of pro- or anti-apoptotic regulatory factors (e.g., Bax or Bcl-2). Thus, when employed in random screening, the cell-based FRET reporter assays developed above are capable of detecting signal transduction inhibitors, e.g., MAP kinase inhibitors, inhibitors of post-translational protein processing, and the like. Because the assay is cell-based any inhibitor so detected is not a priori a protease inhibitor. However, since this particular cell-based assay relies on obtaining a result indicating reversal of a decreasing trend in FRET, (i.e., inhibition of a negative producing a positive result), the possibility existed that significant numbers of compounds might produce false-positive results in the assay. Since screening of compounds using a cell-based FRET reporter assay had not been previously reported, the required performance characteristics of such an assay were unknown. Verification of the assay was thus, on theoretical grounds, problematic, and actual screening data from a random library of compounds was judged to be necessary to confirm its utility as a screening assay capable of identifying inhibitors of apoptosis.

Considering the foregoing, a standardized protocol was adopted, based on the experiments described above, and put to use screening a random library of about 25,000 compounds. As in the EXAMPLES, above, cells were plated in a volume of 100 μl on gelatin-coated black-sided 96 well microtiter plates in 2SM1 medium (Materials & Methods section below). Three to 4 hrs. later plates were checked microscopically to verify normal cell morphology and adherence, and then test compounds, Z-VAD-fmk (10 μM) and vehicle controls (0.25% DMSO) were added followed 1 hr. later by anti-Fas antibody (300 ng/ml). After overnight incubation, FRET fluorescence was determined. As the study progressed and confidence in the assay grew appropriate cutoff values for fluorescence background and toxicity were determined and applied. Initially, all assays were performed in wells on duplicate plates, but eventually when inter-well, inter-assay, day-to-day variability and false hits were all determined to be extremely low, and precision to be very high, only single wells in single plates were subject to testing.

Since many of the compounds in the random library were known to have limited solubility in aqueous solution, 6-fold concentrated (6×) solutions of test compounds (i.e., about 300 μM) were prepared in 2SM1 media containing, (in the 6×solution), 1.5% DMSO then diluted step-wise into the assay to achieve a final concentration of about 50 μM and 0.25% DMSO in the assay.

For convenience, microtiter plates were commonly incubated at 37° C. in 5% CO₂/95% air atmosphere overnight, i.e., about 16-20 hrs., although time-course studies indicated that statistically significant inhibitory effects of certain test compounds could be seen within as little as about 8 to 10 hrs.

After the overnight incubation, fluorescence of the FRET reporter polypeptide in these cells was determined, as described above. To remove any possible background auto-fluorescence of media or test compounds, wells were gently washed about 2 to about 4 times, about 200 μl per well, with PBS (0.1M phosphate-buffered 0.14M saline, pH 7.4). A gelatin-coated plate containing PBS, but no cells, was included in the assay, i.e., for background subtraction. After the final wash, about 200 μl PBS was left in the well. Commonly more than about 85% of the cells remained adherent at this stage. Fluorescence was immediately determined using a Cytofluor II plate reader (Perseptive Biosystems), using a fluorescence source filtered for excitation at 395/40 nm, and detectors set for detecting emission sequentially at 530/10 and 500/10 nm. A total of 10 sequential determinations were recorded for each well. The change in FRET signal, resulting from inhibition of apoptosis, or inhibition of induction of apoptosis, (i.e., inhibition of cleavage of the FRET reporter polypeptide and inhibition of the resultant decrease in fluorescence resonance energy transfer from the exciter fluorophore to the emitter fluorophore), was calculated by taking the ratio of the background-subtracted 530 nm and 500 nm emission values (i.e., the 530:500 nm ratio, supra). Test wells which yielded RFU that were <80% of the mean values recorded in the apoptosis control wells were flagged as being potentially due to toxic effects on the test cells, and test wells that yielded RFU values >200% of mean values recorded in the vehicle control wells were flagged as being potentially due to auto-fluorescence or uncontrolled interference. After certain initial selected flagged compounds were confirmed to be false positive results, all subsequent flagged compounds were just omitted from consideration. The results presented in TABLE VI, show that the FRET reporter assay identified 40 small chemical entities in a library of 24,276 compounds, i.e., 0.16%, that were capable of inhibiting apoptosis. Subsequent studies suggest that 48% of the compounds identified exert some effect on anti-FRET antibody-induced loss of FRET signal.

TABLE VI
Results Obtained in Screening a Random Small Chemical Library

| | |
|---|---|
| Number of Compounds Screened | 24,276 |
| Number of Possible Hits | 63 |
| Number Eliminated Due to Compound Auto- | 23 |
| Fluorescence, Toxicity or Well Position Effects | |
| Number of Actual Hits | 40 |
| Percent Hits of Total Compounds Screened | 0.16% |
| Number of Hits Validated in Subsequent FRET and | 19 |
| Apoptosis-Dose Response Studies- | |
| Percent Validated Hits (% of Total Actual Hits) | 48 |

### MATERIALS AND METHODS

2MM Media: Dulbecco's D-MEM containing L-Glutamine (Gibco #11965-092) and supplemented with 10% Fetal Bovine Serum (Sigma #F2442), 1×Penicillin and Streptomycin (Gibco #15140-015).

2SM1 Selection Media: 2MM media additionally containing 400 μg/ml G418 (Gibco #11811-031).

Gelatin-coated plates: 0.1% gelatin in sterile H₂O was added to each well for 0.5-4 hr., aspirated and then air-dried.

Z-VAD-fmk Control: Z-VAD-fmk (Enzyme Systems Products #FK-009) was prepared as a 40 mM stock in DMSO.

Anti-Fas Antibody: Anti-human Fas monoclonal antibody was purchased having a concentration of 0.5 mg/ml (Upstate Biotechnology #05-201).

Radiation Treatment: Apoptosis was induced by irradiation with UV light, 254 nM, delivered from a Strategene 1800 source.

SYTO13 Cell Viability Assays: Cells were seeded into 96-well plates, incubated overnight to adhere cells to the plastic, and then treated in quadruplicate for various times with extrinsically applied apoptosis-inducing agents, i.e., staurosporin or etoposide. Cell viability was determined using 5 μM SYTO 13 (Molecular Probes) and/or 5 μg/ml propidium iodide (PI, Molecular Probes). As controls, four wells of untreated cells were stained with SYTO 13 and PI, i.e., to measure the maximum signal (max), and four wells of untreated cells were stained with only PI, i.e., to measure the minimum signal (min). After 30 minutes of incubation at 37° C., fluorescence was assayed in plates using the Cytofluor II plate reader (Perseptive Biosystems) (excitation 485/20 nm; emission 530/30 nm; gain 50). Data was collected as relative fluorescence units (rfu) and averaged for six readings per well. Percent cell survival was calculated as 100×(rfuₜᵣₑₐₜₘₑₙₜ−rfuₘᵢₙ)/(rfuₘₐₓ−rfuₘᵢₙ).

Morphologic Markers of Apoptosis: Microscopic markers of apoptosis include membrane blebbing, cell shrinkage (i.e., cytoplasmic condensation) and condensed (dense) chromosomal nuclear materials (i.e., pyknosis).

DNA Fragmentation ELISA: Apoptosis-induced inter-nucleosomal fragmentation of chromosomal DNA was quantified using the “Cell Death Detection ELISA^{PLUS}” (Boehringer Mannheim) according to the manufacturer's instructions. Briefly, ˜10⁴ cells/well were plated on a poly-D-lysine-coated 96-well plate and treated for various periods of times with different apoptosis-inducing agents. The cells were lysed and the nuclei and membranes were pelleted. An aliquot of the cytoplasm-containing supernatant was then transferred to a streptavidin-coated microtiter plate and incubated with anti-histone-biotin and anti-DNA-peroxidase, resulting in the formation of “sandwich” antibody complexes, i.e., (anti-DNA-peroxidase)-(nucleosomal-DNA-containing-histone) -(anti-histone-biotin)-(streptavidin-microtiter plate). Unbound antibody was removed by washing. Peroxidase substrate (i.e., ABTS) was added and nucleosomal DNA complexes quantified by spectrophotometric analysis of the colored peroxidase reaction product.

Apoptotic Membrane Assay: Apoptotic membrane changes (i.e., translocation of phosphatidyl serine from the inner to the outer face of the plasma membrane) were assayed with the ApoAlert AnnexinV kit (Clontech) according to the manufacturer's instructions. Briefly, cells plated and treated as above were washed in 1×PBS, incubated with annexinV-FITC (1 μg/ml) and propidium iodide (50 μg/ml) for 5-15 min, and evaluated by fluorescence microscopy.

Caspase-3 Assay: Caspase-3 activity was assayed according to the following protocol. Cells plated and treated as above were washed in 1×PBS and lysed in 1×assay buffer (1 mM DTT, 1% Triton X-100, 0.5% Tween-20 in 1×PBS, pH 7.2). Lysates were combined with fluorogenic substrate, Ac-DEVD-AMC (200 mM), ±inhibitor, Ac-DEVD-CHO (10 μM) (Bachem) in 1×assay buffer and incubated in the dark for 1 hr. at room temperature. Fluorescence readings were taken on the Cytofluor II plate reader (excitation 360 nm, emission 460 nm, 6 reads/well, gain 50).

Construction of SAT FRET reporters: Plasmids encoding the Trypsin (T) linker polypeptide (SAT) were constructed using PCR to amplify portions of pRSET(A), i.e., from the T7 promoter to the polylinker, and to introduce a NcoI site at the 3′ end of the product, i.e., using oligonucleotide primers T7 and KE76. Plasmids encoding the Apopain (A) linker polypeptide were constructed using synthetic PCR with overlapping oligonucleotides KE76-KE79 to generate the desired linker sequence with an NheI site at the 5′ end and an NcoI site at the 3′ end. The Sapphire coding sequence with an XbaI site replacing the stop codon was amplified by PCR from pRSET(A)-Sapphire using oligonucleotide primers T7 and KE81. An NheI/NcoI restriction fragment of each spacer PCR product was ligated to a 3.5-kb NheI/NcoI fragment of pRSET(A)-Topaz. The resulting intermediate constructs, i.e., pRSET(A)-TT and pRSET(A)-AT, contained, respectively, the T or A spacer 5′ to the Topaz coding sequence. A 0.8-kb NheI/XbaI restriction fragment of the Sapphire PCR product was ligated into the NheI site of pRSET(A)-AT to create pRSET(A)-SAT. The SAT coding sequence was subcloned into vectors pcDNA3 and pcDNA3.1/V5-His (Invitrogen) for expression in mammalian cells.

Construction FRET STT Reporter: The STT reporter links Sapphire and Topaz with a 25-residue trypsin-cleavable spacer similar to the BFP-GFP reporter of Heim and Tsien, 1996. The pRSET(A)-SAT reporter plasmid constructed above contained a trypsin-cleavable spacer linked to Topaz to form an STT precursor, pRSET(A)-TT, described above. The STT coding sequence was subcloned into vector pcDNA3 (Invitrogen) for mammalian expression.

Calcium Phosphate Transfection: Methods for calcium phosphate-mediated transfection of U138 were modified from Sambrook et al. (1989) pp. 16.33-16.35 as follows. Briefly, log phase cells plated about 18 to 24 hrs. before use were washed gently with PBS, released from the substrata with 60-90 s. trypsin (GIBCO) treatment, resuspended in fresh media at a density of about 0.5-3×10⁶ cells/ml and then diluted into 8 ml medium and incubated overnight at 37° C. in 95% air/5-6% CO₂ in tissue culture plates. A calcium phosphate DNA precipitate was prepared for transfection by rapidly adding about 1-20 μg of a plasmid DNA solution in 0.27M CaCl₂/distilled water to an air bubbling solution of 1.4 mM Na₂HPO₄/NaH₂PO₄ buffer containing 0.27 M NaCl and 42 mM HEPES (free acid; Calbiochem), resulting in a final pH of about 6.9. The precipitate formed at room temperature over 20-40 minutes was collected and added dropwise to the plate of cells. After 5-7 hrs. treatment, cell were washed twice with PBS (5 ml each) and fresh media was then added.

Purification of FRET Reporter BFP-GFP Proteins: SAT and STT reporter fusion proteins were purified using Ni-NTA Superflow nickel resin (Qiagen) according to the manufacturer's protocol, with elution in 500 mM imidazole. Selected proteins were further purified using FPLC (Pharmacia; MonoQ HR column; Buffer A=PBS, Buffer B=PBS+1 M NaCl; flow=1 ml/min; original gradient 0-20% B in 20 min, modified gradient 0-10% B in 5 min, 10% B for 5 min, 10-20% B for 5 min; collected 0.5-ml fractions).

Expression of FRET Reporter Polypeptides in Mammalian Cells: Cell lysates were prepared for western blot analysis by homogenizing cells in 50 mM Tris-HCl, pH 7.4. SDS-PAGE was performed using a 4-20% TRIS-glycine gel (Novex) and Laemmli SDS conditions, according to the manufacturer's instructions. Western blots consisted of protein transfer to Hybond ECL membranes (Amersham) according to the manufacturer's instructions and GFP polypeptide fluorophores were detected using GFP-specific polyclonal or monoclonal antibodies (Clontech).

Trypsin Treatment of STT: That cleavage of the STT reporter produced proteolytic cleavage products of the expected size was determined using fractions obtained from FPLC purification of STT fusion protein. The fractions were treated with trypsin (0.2 mg/ml, 20 min, room temperature) and analyzed by western blot using a monoclonal GFP antibody (Clontech). Partially-purified Sapphire fusion protein (Xpress System, Invitrogen) was included for comparison.

Changes in FRET Resulting from Trysin Treatment of STT Reporter: A 23-μl aliquot of each fluorescent fraction of FPLC-purified STT fusion protein was treated with trypsin (0.1 mg/ml trypsin, room temperature, 44 min) and assayed for fluorescence using a Cytofluor II plate reader (excitation 395 nm, emission 495 nm and 530 nm). Partially-purified Sapphire fusion protein (Xpress System, Invitrogen) was included for comparison. The FRET signal plotted was the ratio of background-subtracted 530:495 nm emission measurements for each treatment. The FRET signal decreased over the interval from 0 min to 15 min of trypsin-treatment but remained stable in controls.

Etoposide-Induced Cell Death and Apoptotic DNA Fragmentation in IMR-32 Cells: Confluent IMR-32 cells were treated with 50 μM etoposide phosphate for 2, 8, 17 or 24 hrs. To evaluate apoptosis cells were either: i) stained with SYTO 13 and assayed for fluorescence using a Cytofluor II plate reader (excitation 485/20 nm, emission 530/30 nm), or ii) nucleosome-associated DNA fragments were detected using the Cell Death Detection ELISA^{PLUS} (Boehringer Mannheim) according to the manufacturer's instructions. Etoposide caused a) a decrease in cell survival over 24 hrs., as determined by staining, and in comparison with untreated controls; and, b) an increase in measured apoptotic DNA fragmentation.

Staurosporin-Induced Caspase-3 Activation in HEK293 and IMR-32 Cells: Confluent HEK293 cells were treated with 100-200 μM etoposide for 5, 8 or 24 hrs. or with 1-5 μM staurosporin for 8 hrs. or 24 hrs. Confluent IMR-32 cells were treated with 100 μM etoposide for 8 hrs. Cell lysates were collected and assayed for caspase-3 activity. HEK293 cells treated with 1-5 μM staurosporin for 8 hrs. showed a 6- to 10-fold increase in caspase-3 activity over background but etoposide showed no effect. An 8 hr. etoposide treatment increased caspase-3 activity ˜5-fold in IMR-32 cells.

### CITATIONS

1. Hetts, S. 1998. To Die or Not to Die: An Overview of Apoptosis and its Role in Disease. JAMA 279 (4): 300-307.

2. Cohen,G. M. 1997. Caspases: The Executioners of Apoptosis. Biochem. J. 326: 1-16.

3. Kinloch, R. A., Terherne, J. M., Furness, L. M. and Hajimohamadreza, I. 1999. The Pharmacology of Apoptosis. Trends Pharmacol. Sci. 20: 35-42.

4. Pettmann, B. and Henderson, C. E. 1998. Neuronal Cell Death 20: 633-647.

5. Xu, X., Gerard, A. L., Huang, B. C., Anderson, D. C., Payan, D. g. and Luo, Y. 1998. Detection of Programmed Cell Death using Flourescence Energy Transfer. Nucleic Acids Res. 26 (8): 2034-5.

6. Cubitt, A. B. et al., 1995 Understanding, improving and using green fluorescent proteins. Trends Biochem. Sci. 20: 448-455.

7. Heim, R., Tsien, R. Y. Engineering green fluorescent proteins for improved brightness, longer wavelengths and fluorescence resonance energy transfer. Curr. Biol. 6:178-182, 1996.

8. Heim, R., Cubitt, A. B., Tsien, R. Y. 1995. Improved green fluorescence. Nature 373: 663-664.

9. Pollok, B. A. and R. Heim. 1999. Using GFP in FRET-based applications. Trends in Cell Biology 9(2): 57-60.

10. Goldberg, Y. P., et al. Cleavage of huntingtin by apopain, a proapoptotic cysteine protease, is modulated by the polyglutamine tract. Nature Genet. 13:442-449, 1996.

11. Choi, D. W. 1996. Ischemia-induced neuronal apoptosis. Curr. Opin. Neurobiol. 6: 667-672.

While the preferred embodiment of the invention has been illustrated and described, it will be appreciated that various changes can be made therein without departing from the spirit and scope of the invention.

We claim:

1. A stable recombinant apoptosis reporter cell line expressing a FRET apoptosis reporter polypeptide at a level effective to detect induction of apoptosis by an apoptosis inducer agent, wherein said FRET apoptosis reporter polypeptide comprises an exciter fluorophore peptide bonded with a linker polypeptide that is in turn peptide bonded with an emitter fluorophore, wherein said linker polypeptide comprises a length that is within the Förster radius of said exciter and emitter fluorophores, and wherein addition of said apoptosis inducer agent to a plurality of cells comprising said recombinant apoptosis reporter cell line effects a change in said linker polypeptide that is effective to produce a change in resonance energy transfer from said exciter to said emitter fluorophore.

2. The stable recombinant apoptosis reporter cell line of claim 1, wherein said effective level of said FRET apoptosis reporter polypeptide comprises about 1% to about 3% of the total cell protein of the cells comprising said apoptosis reporter cell line.

3. The stable recombinant apoptosis reporter cell line of claim 2, wherein said effective level of said FRET apoptosis reporter polypeptide comprises about 5μg to about 50 μg of said FRET reporter polypeptide per milligram of total cell protein.

4. The stable recombinant apoptosis reporter cell line of claim 3, wherein said effective level of said FRET apoptosis reporter polypeptide comprises about 20 μg to about 40 μg of said FRET reporter polypeptide per milligram of total cell protein.

5. The stable recombinant apoptosis reporter cell line of claim 4, wherein said effective level of said FRET apoptosis reporter polypeptide comprises about 30 μg to about 40 μg of said FRET reporter polypeptide per milligram of total cell protein.

6. The stable recombinant apoptosis reporter cell line of claim 2, wherein said apoptosis inducer agent is effective to induce apoptosis in greater than about 50% of said plurality of apoptosis reporter cells.

7. The stable recombinant apoptosis reporter cell line of claim 6, wherein said apoptosis inducer agent is effective in said plurality of apoptosis reporter cells to effect a measurable change in an apoptosis assay selected from the group consisting of: a caspase assay, a DNA fragmentation assay, a phosphatidyl serine translocation assay, an assay for mitochondrial energy, an assay for a ribosyl synthetase and an assay for cell viability.

8. The stable recombinant apoptosis reporter cell line of claim 7, further comprising adherent cells.

9. The stable recombinant apoptosis reporter cell line of claim 8, further comprising a plurality of cells among which more than about 80% are capable of retaining an adherent phenotype about 16 to about 24 hours after addition of said apoptosis inducer agent.

10. The stable recombinant apoptosis reporter cell line of claim 9, wherein said exciter fluorophore comprises a polypeptide having an absorbance maximum selected from within the range of about 370 nm to about 490 nm.

11. The stable recombinant apoptosis reporter cell line of claim 10, wherein said emitter fluorophore comprises a polypeptide having an absorbance maximum selected from within the range of about 470 nm to about 515 nm.

12. The stable recombinant apoptosis reporter cell line of claim 10, wherein said exciter fluorophore comprises a polypeptide having an emission maximum selected from within the range of about 440 nm to about 514 nm.

13. The stable recombinant apoptosis reporter cell line of claim 11, wherein said exciter fluorophore comprises a polypeptide having an emission maximum selected from within the range of about 500 nm to about 530 nm.

14. The stable recombinant apoptosis reporter cell line of claim 9, further comprising an F9-1-13 cell line accorded ATCC No. PTA-84 or functional or structural equivalents thereof.

15. A recombinant FRET apoptosis reporter polypeptide comprising an exciter fluorophore polypeptide peptide bonded to a linker polypeptide which is in turn peptide bonded to an emitter fluorophore, wherein said linker polypeptide comprises an amino acid sequence having all of the following in serial array: (i) a first end-cap amino acid domain; (ii) an acidic domain; (iii) a substrate domain; (iv) a non-interferring domain; and (v) a second end-cap amino acid domain.

16. The recombinant FRET apoptosis reporter polypeptide of claim 15, wherein said linker polypeptide comprises about 18 to about 40 amino acids.

17. The recombinant FRET apoptosis reporter polypeptide of claim 16, wherein said linker polypeptide comprises about 18 to about 30 amino acids.

18. The recombinant FRET apoptosis reporter polypeptide of claim 17, wherein said first end-cap amino acid domain comprises about 3 to about 5 amino acids.

19. The recombinant FRET apoptosis reporter polypeptide of claim 18, wherein at least one of said amino acids comprising said first end-cap amino acid domain is selected from the group consisting of Pro, Gly and Tyr.

20. The recombinant FRET apoptosis reporter polypeptide of claim 17, wherein said acidic domain comprises about 2 to about 5 amino acids.

21. The recombinant FRET apoptosis reporter polypeptide of claim 17, wherein said substrate domain comprises about 4 to about 7 amino acids.

22. The recombinant FRET apoptosis reporter polypeptide of claim 21, further comprising an amino acid sequence selected from the group consisting of WEHD, DEHD, DEVD, WEHD, LEHD, WEHD, LEHD, VEHD, DEVD, LETD and LEHD.

23. The recombinant FRET apoptosis reporter polypeptide of claim 17, wherein said non-interfering domain comprises about 3 to about 5 amino acids.

24. The recombinant FRET apoptosis reporter polypeptide of claim 23, wherein said non-interfering domain comprises about 3 to about 7 amino acids.

25. The recombinant FRET apoptosis reporter polypeptide of claim 24, wherein at least one of said amino acids comprising said second end-cap amino acid domain is selected from the group consisting of Pro, Gly and Tyr.

26. A recombinant prokaryotic or eukaryotic cell expressing the FRET apoptosis reporter polypeptide of claim 15.

27. The recombinant eukaryotic cell of claim 26, further comprising a mammalian cell having substantially the same properties as F9-1-13 deposited as ATCC No. PTA-84.

28. A method for preparing a stable FRET apoptosis reporter cell line capable of reporting intracellular apoptotic changes resulting from addition of an extracellular apoptosis inducer agent to a plurality of cells comprising the cell line, wherein said method comprises:
introducing into a plurality of cells a plasmid capable of expressing both a FRET apoptosis reporter polypeptide and a selectable marker,
selecting within the plurality of cells for a cell expressing the selectable marker;
testing within the selected cells for a reporter cell expressing the FRET apoptosis reporter polypeptide at a level of about 1% to about 3% of total cell protein;
adding an apoptosis inducer to a first plurality of said reporter cells and determining that apoptosis is induced in more than 50% of said plurality of reporter cells;
adding an apoptosis inducer agent to a second plurality of said reporter cells, measuring a FRET signal produced by the FRET apoptosis reporter polypeptide in the selected cell and determining that the selected cell is a FRET apoptosis reporter cell if the FRET signal is changed by at least about 14% after addition of the apoptosis inducer agent.

29. The method of claim 28, wherein said FRET apoptosis reporter polypeptide comprises in serial array an exciter fluorophore, a linker region and an emitter fluorophore.

30. The method of claim 29, further comprising the step of determining that a peptide bond in said linker region of said FRET reporter polypeptide is hydrolyzed in the selected reporter cell after addition of the apoptosis inducer agent.

31. A cell line produced by the method of claim 28.

32. A cell line produced according to the method of claim 28 and having substantially the same structural and functional properties in an apoptosis assay as F9-1-13 deposited as ATCC No. PTA-84.

33. A cell-based assay method for identifying and selecting a candidate compound capable of modulating apoptosis induced by an extracellular apoptosis inducer agent, said cell-based assay method comprising the steps of measuring a FRET signal produced by the FRET reporter polypeptide expressed in a recombinant FRET apoptosis reporter cell in the presence and absence of said apoptosis inducer agent and in the presence of said candidate compound, and determining that said candidate compound is effective to modulate apoptosis if: (i) the FRET signal produced in the presence of said apoptosis inducer agent is different than in the absence of said apoptosis inducer agent; and, (ii) in the presence of the combination of said apoptosis inducer agent and said candidate compound the FRET signal produced is substantially the same as the FRET signal produced in the absence of the apoptosis inducer agent.

34. The cell based assay method of claim 33, wherein said FRET apoptosis reporter cell comprises an adherent cell expressing said FRET reporter polypeptide as about 1% to about 3% of total cell protein.

35. The cell-based assay method of claim 33, wherein said intracellular FRET polypeptide comprises in serial array an exciter fluorophore, a linker polypeptide and a emitter fluorophore.

35. The cell-based assay method of claim 35, wherein, said exciter fluorophore comprises a fluorophore having an emission wavelength maximum; said fluorescence emitter fluorophore comprises a fluorophore having an excitation wavelength maximum that is about 10 nm to about 20 nm less than or greater than said emission wavelength maximum of said exciter fluorophore and said linker polypeptide comprising a peptide having about 18 to about 40 amino acid residues.

36. The cell-based assay method of claim 34, further comprising a microtiter assay format wherein about 40,000 to about 250,000 of said cells are effective to produce a measurable change in the FRET signal recorded in said microtiter assay format.

37. The cell-based assay method of claim 34, further comprising a microtiter assay format wherein about 50,000 to about 80,000 of said cells are effective to produce said measurable change in the FRET signal recorded in said microtiter assay format.

38. The cell-based microtiter assay method of claim 36, wherein said measurable change in said FRET signal comprises about a 7% to about a 50% difference between (i) said FRET signal produced in the presence of said apoptosis inducer agent and (ii) said FRET signal produced in the absence of said apoptosis inducer agent.

39. The cell-based microtiter assay method of claim 38, wherein said FRET signal recorded in said microtiter assay format is about 3-fold to about 5-fold greater than background signal recorded in a the absence of said FRET apoptosis reporter cells.

40. The cell-based microtiter assay of claim 38, wherein said measurable change in FRET signal can be recorded at about 10 hours to about 24 hours after addition of said apoptosis inducer agent.

41. The cell-based microtiter assay of claim 38, wherein said measurable change in FRET signal can be recorded at about 16 hours to about 24 hours after addition of said apoptosis inducer agent.

42. The cell-based microtiter assay of claim 38, wherein said measurable change in FRET signal can be recorded at about 16 hours to about 18 hours after addition of said apoptosis inducer agent.

43. The cell-based microtiter assay of claim 42, wherein said apoptosis inducer agent is selected from the group consisting of an anti-Fas antibody, a staurosporin, a TNFα, a TRAIL, a TRANCE, an IL-1, a Apo 3, a glucocorticoid, an okadaic acid, NOC18 and a dose of radiation.

44. The cell-based microtiter assay of claim 42, wherein said apoptosis inducer agent comprises an intracellular proapoptotic agent selected from the group consisting of a Bax, a Bid, a Cifa and a Btf.

45. The cell-based microtiter assay of claim 42, wherein said apoptosis inducer agent comprises deprivation of a life sustaining compound selected from the group consisting of glucose, serum, NGF, PDGF, TGFβ and EGF.

46. The cell-based microtiter assay of claim 42, wherein said apoptosis inducer agent comprises down-regulation of Bcl.

47. The cell-based microtiter assay of claim 43, wherein about 80% to about 95% of said FRET apoptosis reporter cells remain adherent at said about 16 hours to about 18 hours.

48. The cell-based microtiter assay of claim 33, wherein said candidate compound is effective to produce said modulation of apoptosis at a concentration of about 1 μM to about 300 μM.

49. The cell-based microtiter assay of claim 33, wherein said candidate compound is effective to produce said modulation of apoptosis at a concentration of about 10 μM to about 50 μM.

50. A candidate compound capable of modulating apoptosis identified and selected according to the assay of claim 33.

51. The cell-based microtiter assay of claim 33, wherein said FRET signal is measured at a first wavelength and a second different wavelength, the value recorded at said first and said measurements are divided the one into the other thereby to obtain a FRET signal ratio, and said change in FRET signal comprises a change in said FRET signal ratio.

52. The cell-based microtiter assay of claim 51, wherein said FRET signal ratio produced in the presence of said apoptosis inducer agent is about 20% to about 50% less than the FRET signal ratio produced in the absence of said apoptosis inducer agent.

53. The cell-based microtiter assay of claim 51, wherein said FRET signal ratio produced in the presence of said apoptosis inducer agent is about 30% to about 50% less than the FRET signal ratio produced in the absence of said apoptosis inducer agent.

54. The cell-based microtiter assay of claim 51, wherein a measurement of said FRET signal ratio produced in the absence of said apoptosis inducer agent on a first day varies by less than about 10% to about 20% a measurement made under the same conditions on a second day.

55. The cell-based microtiter assay of claim 51, wherein a measurement of said FRET signal ratio produced in the absence of said apoptosis inducer agent comprises a value selected from within the range of about 0.8 to about 1.45.

56. The cell-based microtiter assay of claim 55, wherein a measurement of said FRET signal ratio produced in the absence of said apoptosis inducer agent comprises a value selected from within the range of about 1.27 to about 1.45.

57. The cell-based microtiter assay of claim 51, wherein a measurement of said FRET signal ratio produced in the presence of said apoptosis inducer agent comprises a value selected from within the range of about 0.4 to about 1.05.

58. The cell-based microtiter assay of claim 57, wherein a measurement of said FRET signal ratio produced in the presence of said apoptosis inducer agent comprises a value selected from within the range of about 0.95 to about 1.05.
